(19) United States
(12) Patent Application Publication
(10) Pub. No.: US 2005/0221406 A1
(43) Pub. Date: Oct. 6, 2005

(54) Method, composition and device for treating starch related diseases

(76) Inventor: Tarig Sayed Mustafa Arbab, London (GB)

Correspondence Address: CHALKER FLORES, LLP, 12700 PARK CENTRAL, STE. 455, DALLAS, TX, 75251, US

(21) Appl. No.: 10/930,538

(22) Filed: Aug. 31, 2004

(30) Foreign Application Priority Data
Apr. 5, 2004 (GB) GB 0407583.4

(51) Int. Cl. G01N 33/53; G01N 33/537; G01N 33/543

(52) U.S. Cl. 435/7.92

(57) ABSTRACT

The present invention includes a method of diagnosing an insulin resistant mammal including the steps of obtaining one or more samples for testing; and determining the level of starch in the one or more samples, whereby the presence of starch correlates with insulin resistant.

### TECHNICAL FIELD OF THE INVENTION

The present invention relates in general to the field of treating polysaccharide based diseases and, more particularly to a method, composition and device for treating diabetes.

### CROSS-REFERENCE TO RELATED APPLICATIONS

This application claims priority to U.K. Application No. GB 0407583.4; entitled “Diagnostic Testing And Related Matters” filed Apr. 5, 2004.

### BACKGROUND OF THE INVENTION

Without limiting the scope of the invention, its background is described in connection with Diabetes mellitus. Diabetes is the fourth leading cause of disease-related death in the United States. Diabetes is a chronic disease, which affects an estimated 16 million people in the United States, and worldwide the number grows to more than 125 million people. There are two primary types of diabetes Type I diabetes and Type II diabetes.

Type I diabetes or insulin dependent diabetes accounts for 5 to 10 percent of diabetes in individuals. Typically, in Type I diabetes the body does not produce insulin, therefore requiring the individual to administer regular injections of insulin. Type I often occurs in children and young adults.

Type II diabetes or non-insulin-dependent diabetes is the most common form of the disease and accounts for 90 to 95 percent of diabetes in individuals. Typically, patients with Type II diabetes are unable to make enough, or properly use, insulin to meet the patient's needs. Type II onset often occurs gradually and occurs mainly in people over 40 and especially when the person is overweight. Generally, Type II diabetes can be controlled with the combination of dietary measures, weight reduction and oral medication, however, many individuals ultimately require insulin injections.

In addition to the disease many medical complications are associated with the condition including athreosclerosis, hyperlipidemia, retinal damage, neurological damage, and blindness. What is needed are method of detecting, monitoring and safely and effectively treating patients with diabetes with minimal side effects and without the invasive procedure, such as insulin injection.

### SUMMARY OF THE INVENTION

The present invention is directed to the detection, monitoring and effectively treatment of patients with diabetes and other polysaccharide bases diseases with minimal side effects and without the invasive procedure, such as insulin injection. More particularly, the present invention includes a method of diagnosing an insulin resistant mammal for diabetes, by obtaining one or more samples for testing and determining the level of starch in the one or more samples, whereby the presence of starch correlates with insulin resistant. The method may include the further step of contacting the sample(s) with one or more bicarbonates, one or more alkaline agents, one or more hexokinases, one or more oxidases, one or more amylases or combination thereof. The testing of the sample may include the detection of one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof. The samples may include blood, sweat, tears, urine, saliva, cellular fluids, mucus tissues, skin, organs or combination thereof. If multiple samples are used the samples may be of similar or different types and combinations.

Other embodiments of the present method may include isolating a sample from a patient suspected of having the polysaccharide-based disease and determining the relative level of polysaccharide in the sample. In one example, the patient may be any mammal and even a human. In other embodiments the method may include the determination of the relative level of polysaccharide in the sample that includes the detection of one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof.

Yet another embodiment of the present invention includes a method of treating an insulin resistant patient that includes isolating a sample from a patient suspected of having the polysaccharide based disease, determining the relative level of polysaccharide in the sample and administering a pharmaceutical formulation to the patient that includes one or more hexokinases, one or more oxidases, one or more amylases or combination thereof. The pharmaceutical formulation includes one or more enzymes, one or more compounds, one or more solutions, one or more vitamins, one or more minerals or combinations thereof. In one embodiment the pharmaceutical formulation includes one or more bicarbonates, one or more alkaline agents, one or more hexokinases, one or more oxidases, one or more amylases or combination thereof. The pharmaceutical formulation may be a prepared as an intravenous preparation, a capsule, a tablet, a medicinal syrup, a liquid, a lotion, a spray, an ointment, a cream, a dissolvable tablet, a suppository, an effervescent tablet or combination thereof.

Another embodiment of the present invention includes a diagnostic device. The diagnostic device includes a sensor that is capable of measuring the presence of one or more polysaccharides, wherein the sensor contacts a sample suspected of having polysaccharides present. The sensor detects one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof. In one embodiment the sample used in the diagnostic device may be combined with one or more bicarbonates, one or more hexokinases, one or more oxidases, one or more amylases or combination thereof.

Another embodiment of the present invention includes a method of determining the total sample disaccharide/monosaccharide levels in a patient. The method includes the detection of an initial disaccharide/monosaccharide level in a sample. The one or more oligosaccharides in the sample are converted into one or more monosaccharides. The total disaccharide/monosaccharide level in a sample is detected, whereby the difference between the initial disaccharide/monosaccharide level and the total monosaccharide level is the level of polysaccharides in the sample. The converting of the one or more oligosaccharides to one or more disaccharides or monosaccharides includes adding one or more bicarbonates, one or more hexokinases, one or more oxidases, one or more amylases or combination thereof to the sample.

The present invention includes a pharmaceutical formulation in a dosage form for effectively decreasing polysaccharide levels. The pharmaceutical formulation includes one or more agents capable of converting polysaccharides into disaccharides or monosaccharides. The pharmaceutical formulation is an intravenous preparation, a capsule, a tablet, a medicinal syrup, a liquid, a lotion, a spray, an ointment, a cream, a dissolvable tablet, a suppository, an effervescent tablet or combination thereof. The one or more agents may include one or more bicarbonates, one or more alkaline agents, one or more hexokinases, one or more oxidases, one or more amylases or combination thereof.

The present invention also includes a method of determining a starch level of an individual. The method includes applying a reaction solution to a portion of the skin of the individual. The level of starch in the skin is determined with a sensor that measures the product of the reaction solution on the skin. The sensor may be portable and detect one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof. The reaction solution may include iodine, water, alcohols and salts in various combinations. The alcohol may be alcohols or alcohol mixtures are selected from the group consisting of butanol, methylpropanol, isopropanol, pentanol, methanol, ethanol and hexanol.

Yet other embodiments of the present invention also includes an enzyme in a dosage form for effectively decreasing polysaccharide levels of a patient. The dosage includes a therapeutically effective amount of one or more enzymes capable of converting polysaccharides into disaccharides and/or monosaccharides; and one or more pharmaceutical agents. The enzyme may be of a single type or a blended with one or more different enzymes or a blended with one or more other agents. The enzyme may be one or more hexokinases, one or more oxidases, one or more amylases. Additionally, the enzymes may be from different genus and species.

### BRIEF DESCRIPTION OF THE DRAWINGS

For a more complete understanding of the features and advantages of the present invention, reference is now made to the detailed description of the invention along with the accompanying figures and in which:

FIG. (1) is a depiction of solutions and markers before adding starch;

FIG. (2) is a depiction of solutions and markers after 1 hour and 10 minutes from adding starch;

FIG. (3) is a depiction of solutions and markers after 1 hour and 10 minutes from adding starch and the addition of after adding iodine; and

FIG. (4) is a graph displaying the effect of sodium bicarbonate on exercise.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Diabetes is a medical condition in which the blood sugar level is very high and insulin produced by the body is not produced in an adequate quantity or quality to maintain normal circulating blood glucose. Blood glucose levels rise in diabetics after eating sugars and starch.

Diabetes can lead to severe complications over time, as is well known. These complications are largely due to years of poor glucose control. Diabetes care and complications studies have demonstrated that more frequent monitoring of blood glucose and insulin levels could prevent many of the long term complications of diabetes. However, current blood (e.g., finger-prick) glucose tests are painful, inconvenient due to disruption of daily life, cause fear of hypoglycemia resulting from tighter glucose control and may be difficult to perform in long term diabetic patients due to calluses of the fingers and poor circulation.

All over the world, glucose measurements are done by pricking a finger and extracting a drop of blood, which is applied to a test strip comprising chemical sensitivity to glucose in the blood sample. An optical meter (glucometer) is used to analyze the blood sample and gives a numerical glucose reading. It has been discovered that the glucometer as previously used tests only for disaccharides.

Other types of portable glucometers include a watch glucometer for ionophoresis. This glucometer is an instrument rather like a wristwatch. It applies an electrical current to the skin to take or drive water with dissolved chemicals from the body for testing these outside the body, i.e outside skin. It includes a form of spectrophotometer.

Understanding the importance and role of saccharides (Carbohydrates) in maintaining body sugars levels and energy supply. Monosaccharides (simple non-complex sugars) are mainly an immediate source of energy needed at tissue and cells level. Disaccharides (slightly or moderately complex sugars) are always there in the blood stand by (immediate blood sugar reserve) awaiting quick conversion into simple sugars when needed usually after the utilization of simple sugars due to activity. Part of ingested disaccharides (sucrose, lactose etc), get initially prepared for absorption by amylase in the mouth and then absorbed directly into the blood as Disaccharides. The action of amylase on disaccharides (e.g., amylase disaccharides phenomenon) was never known or documented before. The rest of disaccharides pass into the stomach and intestines to get metabolized by enzymes like sucrase and lactase in the intestines and absorbed as simple sugars (monosaccharide) into the blood. Polysaccharides are the main body reserve that supplies Disaccharides regularly to maintain the immediate blood sugar reserve (amylase convert polysaccharides into disaccharides).

Amylase is an enzyme that changes complex sugars (starches) into simple sugars during digestion. There are many types of amylase secreted by various tissues in the body including blood cells. There are two main kinds of amylase enzymes: Alpha-amylase and Pancreatic amylase. Alpha-amylase (ptyalin), which is produced by the salivary glands. This enzyme begins starch digestion in the mouth and continues to work in the stomach.

Pancreatic amylase, which is secreted by the pancreas into the small intestine. This amylase continues the starch digestion process. Levels of amylase in the blood can be used to help diagnose and monitor diseases, such as diseases of the pancreas and salivary glands, or to determine whether the intestines have been damaged.

Starches are insoluble in water and thus can serve as storage depots of glucose. Raw starch can be deposited in blood (blood walls and circulating blood), and tissues. Starch deposit on blood walls attracts calcium, fibers, lipids and other materials in the blood including blood cells to be deposited on it and forms what so called atheroma. Starch granules usually absorb water into them and increase in size; in addition to that starch has a property of gelatinisation (forming gel).

Before starch can enter or leave cells, the starch must be digested or hydrolyzed. The hydrolysis of starch is done by amylases. With the aid of an amylase (such as salivary and pancreatic amylase), water molecules enter at the 1-4 linkages, breaking the chain and eventually producing a mixture of glucose and maltose. However, a different enzyme is needed to break the 1-6 bonds of amylopectin.

Diabetes is usually associated with a high presence of raw, non-degraded disaccharides (sucrose, lactose, etc) and raw, non-degraded (uncooked) polysaccharides (starch) in the blood. Monosaccharides are the main body fuel resulting from the metabolism of all sugars this is normally processed and utilized immediately by the body for energy. Insulin is an effective hormone that drives monosaccharide and potassium into tissues and cells. Insulin also acts on disaccharides by releasing glucose from it is molecules and drive it to tissues and cells. Insulin is less effective or perhaps non-effective on polysaccharides due to their tight molecular bonds resulting in an insulin resistance. In this case sodium bicarbonate is very helpful in destabilizing starch molecules and loosening their tight bonds. Sodium bicarbonate help the insulin to penetrate the unstable compounds of polysaccharides and act directly on molecules to free glucose, in addition to that, Amylase and other enzymes will act as catalyst to help convert polysaccharides into disaccharides. Amylase react with all sugars namely disaccharides, polysaccharides and probably monosaccharides and others in a very similar way like that of the insulin, but amylase is slower taking from few hours to days to decrease blood sugar levels.

Type I is characterized by high blood levels of disaccharides (mainly due to high disaccharides intake), which will require high doses of insulin to be given. Type I appears to affects younger people because they are difficult to control in respect to disaccharides intake, which normally present in sweets, soft drinks, etc. Type I, appears to affect young persons because they do not control their disaccharides (e.g., sucrose, lactose, cellulose, etc) intake.

Type II usually due to high intake of polysaccharides, and it affects adults because they can control them self from high disaccharides intake. Type I if complicated with high polysaccharide intake blood sugar levels will be difficult to control. It will be a mixture of Type II which is normally caused by excess intake of polysaccharides and type one which is mainly caused by excessive intake of disaccharides), and in this case, the insulin may become less effective or non effective in some cases (insulin resistance). Type II is mainly due to high polysaccharides intake, which will not require insulin to be given unless this is complicated by high disaccharides intake, then high doses of insulin will be required.

Both Type I and Type II if complicated with long standing high polysaccharides intake, then insulin will be required regularly and both types become one type (Type I). This probably due to the deposit of polysaccharides in tissues (like pancreas, liver, kidneys, lungs, heart, brain and skin) and specifically small arteries (microcirculation) are blocked by starch. As a result insulin can not be produced due to tissue alteration and malfunctioning caused by starch deposit, which will lead to tissue and cell ischaemia, necrosis, and calcifications with cell death. At the same time insulin can not be released in blood because of blocked arteries with excessive deposit of starch (insulin as a hormone gets released directly in blood stream).

Conclusion: Type I diabetes is associated with high disaccharides intake and high disaccharides levels in the blood. Therefore, insulin is needed to reduce disaccharide levels. Type II diabetes is associated with high polysaccharides intake. Therefore, oral tablets are needed to reduce polysaccharides levels. Acute Type I and Type II diabetes are associated with high disaccharides intake consequently high disaccharides levels in the blood in addition to high polysaccharides intake and consequently high polysaccharides levels in the blood. Therefore, insulin and oral treatments are needed temporarily. Chronic Type I and Type II diabetes are associated with a high disaccharides intake level and a high level of disaccharides in the blood, in addition to a high polysaccharides intake level and a high level of polysaccharides in the blood. Therefore, in chronic Type I and Type II diabetes insulin treatment and oral treatments are needed continuously.

There are forms of diabetes that do not respond to insulin treatment associated with starch in the blood. Insulin served to help the body utilize the sugar, when this is a monosaccharide by breaking the sugars down to provide energy. In forms of diabetes that do not respond to insulin treatment the diabetic patient eats sugar and the blood glucose raises, and eating starch produces the same result. Thus, starch is equivalent to sugar. There is normally a chain of chemical conversions, from polysaccharides (starch) through disaccharides to monosaccharide to energy. It has been shown that, with these forms of diabetes, there is zero or insufficient conversion of the polysaccharide to the disaccharide, and there is starch polysaccharide in the blood stream and tissues. Additionally, the presence of starch in the blood is associated with certain other diseases, e.g. alzheimer's disease, arthritis, asthma, high cholesterol levels, obesity, retinopathy, microangiopathy, amyloidosis, D.V.T (deep vein thrombosis), renal failure, organ failure, leg ulcers, and epilepsy; and that measurement of the starch can give an indication of these diseases (i.e. measurements are tests which can help a diagnostician to diagnose these disease and or their seriousness). Measurements of starch in blood can also help prevent (prophylaxis) diabetes complications, and all other diseases associated with presence of starch in the blood. Measurements of the starch in blood can also be used in sport and exercise activity to control and enhance performance. Additionally, it is useful to measure polysaccharides in blood. It has been discovered that, in other diseases, there is a starch deposits on the walls of the bloods vascular system, usually the arterial system and tissues i.e. undigested raw starch blocking the arteries, including the pancreatic arteries, with deposit on pancreatic tissue, and preventing insulin from reaching the blood or the skin.

Diabetes may thus be due to inadequate secretion of insulin (e.g. if due to blocked arteries) or inadequate utilization of insulin (e.g. no reaction to disaccharides). The present invention helps, treats, or prevents theses diseases through the reduction of the starch could prevent.

It has been shown that starch can be converted to disaccharide by use of amylase, often greatly helped by use of sodium bicarbonate, or by use of hexokinase or oxidase, or by cocktail of enzymes, both for diagnosis and treatment. The term “treatment” herein means giving temporary relief and or a permanent cure (as the case may be). The sodium bicarbonate action discovered by the inventor is that it does itself destabilize the polysaccharide into smaller molecules that makes it easier for the amylase to convert into disaccharide.

Previously insulin was given to treat diabetes; it only treats some kinds of diabetes; the present inventor realized that it only treat excess monosaccharide. Diabetes may not be the direct result of a lack of insulin. The presence of undigested raw starch can block the arteries. Some embodiments of the present invention includes administering amylase, alone or in combination with other enzymes as part of a test and treatment, in instance where there is insufficient conversion from polysaccharide to disaccharide and monosaccharide.

According to the broad aspect of the invention, there is provided the utilization of these discoveries, namely the presence of starch in the blood stream and or blood vascular system and tissues and it is relationship to various diseases, for testing, diagnosis and or treatment/prophylaxis. This includes methods of testing, treatment and prophylaxis, substances for testing, treatment and prophylaxis, and instruments for testing and treatment.

Sodium bicarbonate (NaHCO₃) ingestion has been shown to increase both muscle glycogenolysis and glycolysis during brief submaximal exercise. These changes may be detrimental to performance during more prolonged, exhaustive exercise. This study examined the effect of NaHCO₃ ingestion on muscle metabolism and performance during intense endurance exercise.

High-intensity exercise, at or near 100% of CO₂ max, can be maintained for only a matter of minutes, and the cessation of such exercise is generally associated with muscular fatigue and discomfort. Muscular fatigue resulting from high-intensity exercise is due in part to a decrease in intramuscular pH. The metabolic demands of high-intensity exercise are met primarily by glycolysis, which is the non-oxidative breakdown of glucose resulting in the production of lactic and other metabolic acids which decrease the pH of exercising muscles. The onset of muscular fatigue is associated with a rapid increase in the production of these metabolic acids, and tolerance of high-intensity exercise may be limited by the body's ability to counteract decreases in intracellular (muscle) and extracellular (blood and interstitium) pH through its intrinsic buffering systems. For many years coaches and trainers have recommended the use of sodium bicarbonate (NaHCO₃) to increase exercise performance, and numerous studies have examined the effects of increased buffer capacity on acid-base balance, endurance, and power output.

During maximal exercise blood and muscle lactate increase dramatically. While there is considerable debate as to whether this represents anaerobiosis, or the lack of oxygen, it is generally accepted that increased blood and muscle lactate are a result of an increase in glycolysis. The increased dependence on glycolysis during exhaustive exercise results in altered acid-base balance, and the concurrent increase in production of lactic and pyruvic acids results in increased intracellular and extracellular hydrogen ion concentration [H⁺], which is buffered by the bicarbonate ion (HCO₃⁻) system as depicted in the following equation:

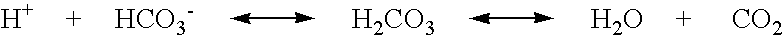

During high-intensity exercise intracellular and extracellular lactic acid concentrations increase as a function of exercise duration and, similarly, bicarbonate ion concentration decreases during high-intensity exercise. Associated with these metabolic changes is a decrease in pH that is related to muscular fatigue. A positive linear correlation has been shown between hydrogen ion concentration in muscle and muscle fatigue. Furthermore, recovery of fatigued muscles is associated with the removal of lactate and hydrogen ions from muscle cells. Thus, it has been hypothesized that increasing the body's buffering capacity (i.e. increasing the amount of circulating HCO₃⁻) would protect against acidosis and thereby delay the onset of muscle fatigue during exercise.

It has been shown that sodium bicarbonate enhances sport activity mainly due to destabilization of starch in blood and tissues which then makes it easy for insulin and amylase to break down the polysaccharides bonds and liberate glucose, rather than what has been always claimed to have been caused mainly by anaerobic metabolism of muscles.

It has also been shown that the basis of the use of exogenous sodium bicarbonate (NaHCO₃) as a method of increasing buffering capacity and destabilizing polysaccharides molecules which ease polysaccharides molecular bonds to liberate glucose by insulin or amylase, delaying the onset of fatigue, and increasing exercise performance.

Polysaccharides aerobic Glycolysis as an Energy Source: During exercise tension is generated within a muscle by the formation of a protein complex called actomyosin. This complex is formed from two myofibrillar proteins (actin and myosin), and requires energy in the form of adenosine triphosphate (ATP):

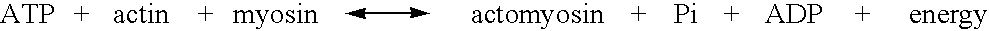

Where Pi is inorganic phosphate and ADP is adenosine diphosphate. ATP is provided from two energy sources: immediate, and oxidative. The immediate energy sources include stored ATP, creatine phosphate (CP), and ADP. Together, these high-energy phosphates provide energy immediately upon demand but are exhausted within thirty seconds.

Oxidative body energy is generated from the oxidation of polysaccharides (crude energy that is to say raw uncooked starch) that found stored in blood stream and body tissues. A relatively rapid supply of energy can be provided to working muscle in the presence of oxygen (O₂) by the following equation termed polysaccharides aerobic glycolysis: 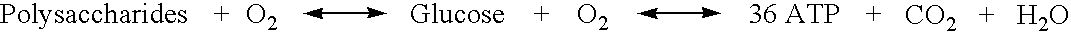
or polysaccharides aerobic ergogenic aided glycolysis:

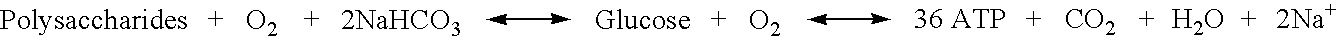

Although this aerobic glycolysis is capable of providing energy rapidly, it cannot sustain high power output activity for more than two minutes. In contrast, the oxidative catabolism of glucose or fatty acids below can provide a wealth of ATP:

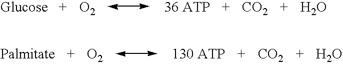

The oxidative energy system provides energy for sustained (endurance) activities, but involves numerous complex enzymatic pathways and cannot provide ATP as rapidly as the immediate and oxidative energy systems (crude energy sourced from blood and tissue stored free polysaccharides). During high-intensity exercise lasting 1 to 7 minutes, oxidative and stored polysaccharides oxidative energy sources both provide ATP, but the high power output of this type of activity is primarily met by polysaccharides aerobic glycolysis. Increased polysaccharides aerobic glycolysis contributes in the formation of lactic acid, which dissociates at normal physiological pH to lactate and free hydrogen ion (H+). It is the generation of hydrogen ions from the production of lactic acid that is primarily responsible for decreasing intramuscular pH during high-intensity exercise. The resulting shift in acid-base balance negatively together with rapid utilisation of all sources of body energy generated from free glucose and polysaccharides affects energy production and contractile function, and may be responsible for cessation of exercise.

Blood and Muscle pH during exercise: At rest arterial blood pH is about 7.4, while venous blood pH is normally slightly lower (between about 7.3 and 7.35) and muscle pH is about 6.9. Exhaustive exercise decreases pH about 0.4 pH units in both blood and muscle, and is highly correlated to increased blood lactate concentration. Similarly, blood and muscle bicarbonate ion concentration decreases linearly as a function of increasing lactate ion concentration.

Post-exercise blood lactate increases as a function of exercise duration or distance up to 1500 m, with no further increase following a 5000 m run. Similarly, pH and bicarbonate ion concentration were lowest after a 1500 m run. These data indicate that acid-base shifts occur most dramatically during exercise that can be sustained less than 7 minutes. This is due to the fact that exercise resulting in exhaustion beyond 5 minutes recruits fewer Type II (fast glycolytic and fast oxidative glycolytic) fibers, is less dependent upon polysaccharide oxidative energy sources, and results in a lower blood lactate response. This is an important point to consider when reviewing exercise protocols used to test the effectiveness of NaHCO₃ as an ergogenic aid.

Similar to blood lactate, muscle lactate is elevated and pH is decreased following exercise. Several reports indicate that resting blood pH is significantly greater and maximal exercise endurance greater in subjects who ingested NaHCO₃ prior to exercise. Because intramuscular pH is not different at exhaustion between placebo and NaHCO3 treated groups, it might be concluded that intramuscular pH is not a limiting factor during maximal exercise. However, subjects who are administered NaHCO₃ prior to exercise are able to sustain near maximal to supramaximal exercise for a longer period of time. Thus, while intramuscular pH was not different between treatments, the administration of NaHCO₃ may have delayed the onset of critically low muscle pH associated with fatigue. Furthermore, as will be discussed in the following section both contractile function and non-oxidative energy production are sensitive to low intracellular pH.

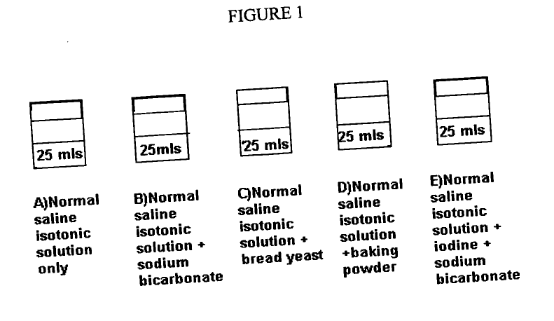

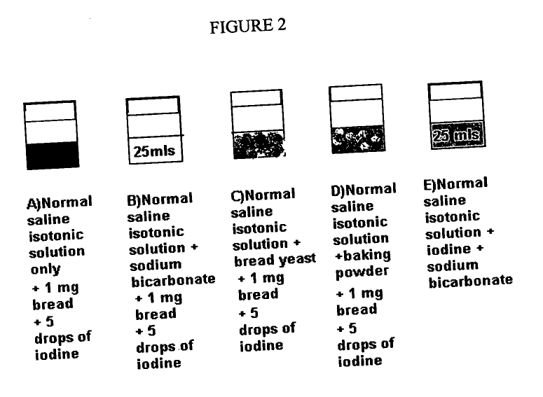

Material and Methods: In Vivo Test example: Fifteen healthy volunteers, different age groups varies between 20 to 32 years, body weight between 65 to 70 kg, male subjects, were put for exercise test under three conditions: following ingestion of 300 mg sodium bicarbonate per kg of body mass (i); following ingestion of a placebo (100 mg sodium chloride isotonic solution per kg of body mass) (ii); and following ingestion of neither (iii). A double-blind protocol was used between the (i) and (ii) trials. All volunteers had a starchy meal before going to bed the night before exercise. Each condition was repeated so that the volunteers underwent treadmill exercise for six times. 100 minutes before commencing treadmill exercise was allowed after ingesting substances in (i), (ii) and (iii). The volunteers exercised until fatigue. Fourteen of the volunteers completed all the tests. The timing of bicarbonate ingestion is also an important consideration. Most investigators have reported that subjects ingested NaHCO₃ between 1 hour and 3 hours prior to exercise. FIG. 1 represents the typical time course of increased venous blood pH and bicarbonate ion concentration following ingestion of NaHCO₃. Maximal rise in pH bicarbonate ion concentration is achieved about 1.5 hours after ingestion, and remains elevated at least 2 hours after ingestion.

One embodiments of the present invention includes an amylase enzyme test, which is specific for starch, for testing, diagnosis and treatment. The amylase on a strip to test a blood sample in-vitro, or placed into the blood stream first and then tests for the resulting disaccharide with glucometer. One embodiment of the present invention uses amylase on a strip. A glucometer is used to detect the amount of glucose produced by the amylase. The glucometer can be re-calibrated to account for the color difference with respect to detection or comparison. Amylase is secreted by the pancreas and other organs which secrete insulin, so that a diabetic who has ineffective or insufficient insulin will have likewise ineffective amylase and therefore too much starch in the bloodstream.

A second test. In the “Arbab test.” named after the inventor, instead of amylase, sodium bicarbonate is used to break down starch. The sodium bicarbonate is given as a drink and one hour later measures the sugar (disaccharide or monosaccharide) as before.

A third test. As with the second test, is non-invasive, e.g., there is no need to break the skin. In this test an iodine solution is contacted with the skin. In some embodiments the iodine solution can contain iodine as about a 0, 1, 3, 5, 7, 9, 10, 11, 13, 15, 17, 20, 22 or 25% w/w solution in alcohol used in a hypertonic solution of sodium chloride (used as a vehicle to facilitate diffusion (dialysis) of the iodine into the skin) to detect starch. The solution remains in contact with the skin for about 15, 30, 45, 60, 75, 90, 105, 120, 135, 150, 165, 180, 195 or 210 minutes. The solution is soaked into cotton pad; this is then put onto the skin, and held in position by means of a neutral patch for between one and 5 hours. Blood sugar levels greater than 20 mmol per liter, can be discerned with the naked eye the consequent blue patch appearing on the skin. Preferably, the color is read with a calorimeter, e.g. rather like watch in size and manner of attachment to the wrist, which uses infra-red spectrophotometer, and can readily detect whether the starch present is in the range of about 0 to 2 mmol per liter for a normal person or greater than 2 mmol per liter for a diabetic. However, other optical analyzers may be used. This embodiment uses diffusion osmosis into the skin for the iodine. The first application of the pad produces a stain effective for up to about five days and can be read continuously by the colorimeter if this has the described watch-like form. The colorimeter can also be arranged to act as a recorder and/or a transmitter. Prior to this starch from the body had not been measured for any purpose.

Another embodiment of the present invention is a colorimeter that fits on the body, e.g. like a watch, necklace, belt, fanny pack, book bag, purse, arm band, or integrated into the clothing of the individual. The apparatus reflects light containing color, passing through a prism to sense the presence and intensity of light in relevant parts of the spectrum using a standard infra-red spectrophotometry used in a glucometer without the electric current to drive out fluid. Another embodiment of the present invention includes the testing of the level of starch in blood or urine using iodine strips.

Methods of testing for starch in the body: Microscopic: Blood or Urine (after centrifugation of the sample). Microscopic reading can be done on the obtained serum, retained centrifuged products (red cells with polymorphic and other blood abstracts) or from whole blood.

Blood Sample: Obtain blood sample of between about 4 to 5 mls. Check the glucose levels of the sample which may be centrifuge or not (e.g., sample can be serum, cells or whole blood). Amylase is added to the sample and the blood glucose is checked hourly for 24 hours. For faster results the sample can be heated. Urine Sample: Obtain a urine sample of about 4 to 5 mls. The level of glucose is checked and the sample centrifuge. Amylase is then added to the sample and the urinary glucose levels checked hourly for 24 hours. The sample may be heated at less than 60° C. degrees.

Examples of new strip testing methods embodying the invention: Use normal glucometer. Starchometer. Gluco-starchometer. Iodo-starchometer. Strips:

Examples of new strip testing methods embodying the invention: Use normal glucometer. Starchometer. Gluco-starchometer. Iodo-starchometer. Strips: Normal blood glucose strip; Check blood glucose levels. Administer a cup of sodium bicarbonate to the patient (quarter of teaspoon in 170 to 200 mls. of water) or 300 mg of sodium bicarbonate to the patient. Check the glucose levels after one hour, using glucometer. Results: The initial glucose levels checked in added to the glucose levels checked after one hour from ingestion of sodium bicarbonate yield the complete glucose levels in the blood. (e.g., Glucose levels initial and Starch (converted to glucose)=Complete glucose levels in the blood).

Blood starch strip: A strip comprising amylase which is sensitive to starch (converts starch into glucose), is measured using glucometer. The blood starch strip that include amylase: A spectrophotometer machine used as a starchometer will be needed to read the results. The sample can be heated to increase the reaction rate. The blood strip with hexokinase or oxidase, or peroxidase plus amylase may be used on a normal glucometer, which thus serves as a glucostarchometer.

Blood iodine strip. Require: a blood drop is added to the sample, or a centrifuged blood sample is used. The results can be read using spectrophotometry machine, which therefore acts as a starchometer but serves as an iodometer.

Urine Glucose strip: Normal testing strip: check urine glucose levels in a sample using normal glucometer. Amylase is added to the sample and the glucose levels rechecked with normal strip, using glucometer. The sample can be heated to increase the reaction rate. Urine strip. Normal existing urine strip for testing glucose and amylase producing results. The results can be obtained using spectrophotometer, which therefore acts as a glucometer but serves as a glucostarchometer.

Urine starch strip: the strip includes amylase which is sensitive to starch (e.g., converts starch into glucose). A spectrophotometer machine as starchometer will need to read the results. The sample can be heated to increase the reaction rate. Urine dip strip. Requires: A urine sample or urine centrifuged or boiled urinary starch can be detected after boiling or centrifuging, then the results can be read using spectrophotometer machine, which therefore acts as a an starchometer but serves as iodometer. Interpretation of the results for iodine strip tests for urine can be read as (+ve=mild) or (++ve=moderate) or (+++ve=large).

Examples of non-invasive technique, using diffusion and osmosis (considering skin as a membrane) measuring starch. Basics: An iodine diffusible solution containing: Iodine 2, 5, 7, 10 12, 15, 17 or 20%; sodium chloride solution (hypertonic); ethanol; warm solution; is added to a cotton skin patch. The skin is then observed with a optical meter. The optical meter can be a watch like reading meter with infrared spectrophotometer. Continuous reading for up to between about 2 to 5 days The time will depend on the amount of starch deposited in the skin.

Method: Cotton skin patch soaked with a iodine solution is applied to the skin surface of the skin for about 1 to 3 hours. using a A spectrophotometer contacted with the forearm or wrist area is used to monitor the changes of color on the skin. The skin is monitored for between 1 to 4 days. Results: A blue color is produced. The color may be visible to the naked eye when glucose levels are above 20 to 25 mmol per liter or more. The color may persists for 2 to 4 days, or even to 5 days. If the reading is low, the starch levels are low, and also the sugar levels will be low. No reading, may mean no starch deposited in the skin, which can also mean low, normal or near normal glucose levels. Note that the blue color cannot be seen all the time by the naked eye and specially if the blood sugar is below 20 mmol per liter.

Another embodiment of the present invention is a non-invasive technique, using hexokinase, peroxidase or oxidase. hexokinase, peroxidase or oxidase and sodium chloride (e.g., hypertonic solution and ethanol in a warm solution, using a glucometer.

Another embodiment of the present invention is a non-invasive technique providing a starch tolerance test. The test involves an initial fasting stage followed by a starchy (e.g. potato) meal. Followed by fasting overnight. The glucose levels are checked in the morning. A 100 mg portion of starch is digested. The glucose levels are checked in half-hour, one hour, and two hours. Raised glucose levels will indicate early diabetes (normal glucose reading should be 0 to 3 mmol per liter).

Yet another embodiment of the present invention is a non-invasive technique, providing a starch-glucose test “The Arbab Starch-Glucose Test”: initially no sugar or sweets are administered for 24 hours prior to the test. The patient then has a starchy meal before going to bed, followed by an overnight fast. The glucose levels are checked before bed and in the morning. The patient then take a quarter teaspoon of sodium bicarbonate in 170 ml of water, or one eighth, one half, three fourths, or one teaspoon of sodium bicarbonate in 170 ml of water. Additionally the drink may be between about 150 ml and 1000 ml of sodium bicarbonate drink in the morning. The glucose levels are then checked after half hour, one hour, and two hours. Results: Raised sugar levels are indicative of pre-diabetes. This is compared with people having a normal sugar level which is raised by this test by, only 2 to 3 mmol per liter (normal range 0 to 3 mmol per liter).

One embodiment of the present invention includes a method of treating diabetes: The method includes checking the patient glucose levels, before starting treatment. Checking the HbA1C levels, before starting treatment and checking the blood tests (kidney function tests) before starting treatment. The patient is given a drink of soda water (quarter teaspoon sodium bicarbonate in 170 to 200 ml of water, or ready made soda water from the supermarket 500 ml or one liter), once a day for three days. Checking the blood glucose levels one hour after drinking soda water (glucose levels will be raised). The next step is to administer to the patient amylase capsules 50 mg, or 25, 30, 35, 40, 45, 50, 55, 60, 65, 70, 75, 80, 85, 90, 95, 100, 105, 110, 115, 120, 125, 130, 135, 140, 145 and 150 mg, to be taken three times a day. Alternatively, pancreas intestinal replacement therapy capsules (P.I.R.T) may be given three times a day, preferably before meals (the amount of amylase depending upon the amount of starch in the meal). Administering medication for diabetes (insulin or tablets) to the patient. Followed by checking the blood and urine for glucose three or more times per day. The process is repeated. Checking the HbA1C levels each day. Results: The HbA1C level may be reduced by 2 to 7% from the initial reading or back to near normal or normal (normal range 4 to 6%). The insulin or tablets may be reduce in dosage as the patient is diabetes indication free.

Tests and Methods: In Vitro: Material and Methods: For the Sodium bicarbonate test these and other materials may be used to detect starch: Specimen pot with lid; Small piece of bread about 1 to 2 mg in weight; Isotonic solution of normal saline about 25 mls; Iodine tincture; Sodium bicarbonate powder; Bread yeast; Baking powder; Plain flour; and a watch or chronometer for timing.

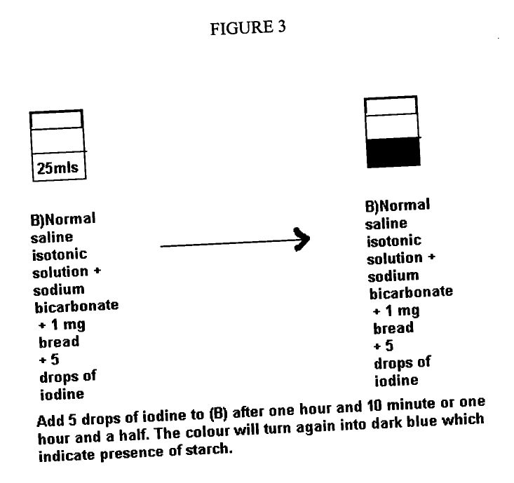

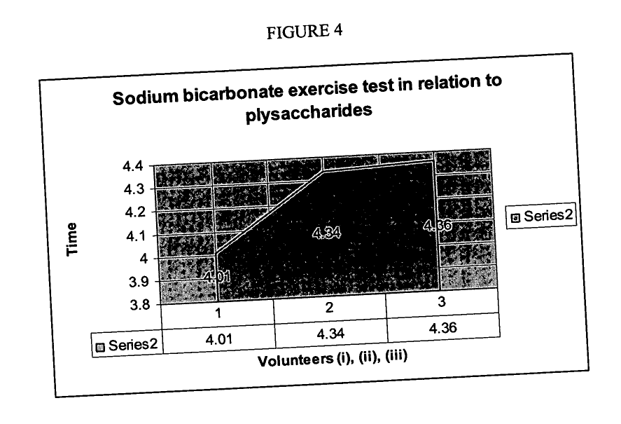

Method (bicarbonate): As depicted in FIGS. 1, 2 and 3. FIG. 1 depicts the samples A a normal saline isotonic solution, B a normal saline isotonic solution and sodium bicarbonate, C a normal saline isotonic solution and bread yeast, D a normal saline isotonic solution and baking power, and E a normal saline isotonic solution and iodine and sodium bicarbonate. At a certain time 1 mg of bread or flour is added to each solution in A, B, C, and D. Iodine tincture, about 5 drops, is added to each specimen pot and the sample shaken well. A dark blue coloring of bread or flour will appear immediately indicating presence of the starch. Each specimen pot sample is shaken about every 15 to 20 minutes. Read and record results in each specimen pot about every 15 to 20 minutes for one hour and a half. More iodine drops (about 5 drops to each specimen pot) were added to each specimen pot after one hour and a half and the results recorded. Results: Specimen pot (A), no change in color for the past one hour and a half. Specimen pot (B), color changed gradually from blue to purple and slight purple, then rose (pink) like color and finally to colorless after about one hour (60 to 70 minute) and bread color return to normal. FIG. 2 demonstrates the samples after 1 hour and 10 minutes (or one hour and a half) from adding starch (A & E are control markers) colors changes in sample B, C, D as illustrated below. Starch in sample (B) returned to it is normal color with colorless sample. Specimen pot (C), color changed partially from blue to purple with still persistence of blue coloration; (bread will be partially dark blue coloring with purple like patches discolorations). Specimen pot (D), color changed partially from blue to purple with still persistence of blue coloration; (bread will be partially dark blue coloring with purple like patches discolorations).

Adding more iodine drops (about 5 drops) to each specimen pot will result in dark blue discoloration in specimen A, B, C. This indicates that starch still present in each sample and has not gone away or degraded despite returning back to normal color with colorless sample in specimen pot B. FIG. 2 demonstrates the samples after 1 hour and 30 minutes and the color in sample (B) become clear, but blue again after adding iodine. (Sodium bicarbonate destabilized starch molecule after one hour and 10 minutes, but starch still present).

Interpretation of results: Results showed: in specimen pot (B), indicates that sodium bicarbonate has destabilized the starch (polysaccharides), molecule and changed its characteristics and properties but did not convert or degraded starch into disaccharides or monosaccharide. The same in vitro reaction between sodium bicarbonate and starch (polysaccharides) takes place in human body and blood. Starch granules are leached out of tissues including blood by sodium bicarbonate.

Material and Methods: One or more of the following are used in the Amylase test for normal and diabetic subject: The materials included specimen pots with lid; small piece of bread 1 to 2 mg in weight or flour; isotonic solution of normal saline 25 mls; Amylase normal healthy human saliva 25 mls; amylase diabetic Type I human saliva 25 mls; amylase diabetic Type II human saliva 25 mls; iodine tincture; sodium bicarbonate powder; blood glucose strips and glucometer; urine glucose strips; and a watch or chronometer for timing. Method: Iodine Test: Put an amount of 25 mls. of normal healthy subject saliva in a specimen pot (A); Put 25 mls. of Type I diabetic patient saliva in specimen pot (B); Put 25 mls of Type II diabetic patients in a specimen pot (C); Pot a small piece of white bread 1 to 2 mg in each specimen pots A, B and C; Time to be recorded; Put iodine drops (5 drops), in each specimen pot immediately; Record change of color.

The iodine test was repeated every 5 minutes for half and hour and record any change in color in each specimen pot. The iodine test was repeated about every one hour after the first half an hour and for 2 hours and record any change in color. The iodine test was repeated, in each specimen pot 4 hourly for 24 hours and record any change in color.

Results: Iodine Test Results: In specimen pot (A), there was immediate color reaction when iodine is added. The piece of bread was stained with dark blue color (blue color noted also, on some parts of the bread and the solution). In specimen pot (B), there were no immediate changes in color for the bread and the solution. The iodine yellow brown color persisted without staining the bread, and visually detect that diabetic Type I saliva (amylase) prevents the reaction between iodine and starch (polysacharides). The polysaccharides were shielded from iodine stain reaction. No color reaction between iodine and bread was recorded for approx 20 to 30 minutes. After 20 to 30 minutes a stain reaction developed gradually and slowly. In specimen pot (C), there were no immediate changes in color of the bread or the solution. The iodine yellow brown color persisted without stain reaction with polysaccharides or the solution and again diabetic Type II saliva (amylase) may be visualized, prevented immediate stain reaction between starch and polysaccharides. No color reaction between iodine and bread was recorded for approximately 10 to 15 minutes. After about 10 to 15 minutes a stain reaction developed gradually and slowly. Final iodine test carried out that is to say after 24 hours recorded no color reaction between iodine and bread and the iodine yellow brown color remained unchanged for approximately one hour then the solution and the bread become stain free again.

Glucose Test: Check glucose levels in each specimen pot every half an hour for the first 2 hours, then every 4 hours for 24 hours (measurement to be done using normal glucometer and urine dip stix for glucose). Check the final glucose levels in each specimen after 24 hours and record the results.

Glucose Test Results: Glucose test results were obtained after 24 hours from each specimen pot and the result were as follows: In specimen pot (A), glucometer tested positive for glucose 7 mmols, and urine strip test was positive for glucose only one cross (+). In specimen pot (B), glucometer tested positive for glucose 16 mmols, and urine strip test tested positive for glucose two crosses (++). In specimen pot (B), glucometer tested positive for glucose 10 mmols, and urine strip test tested positive for glucose one cross (+).

Interpretation of results: Amylase reacts with starch (saliva amylase or plant, etc), to convert starch (polysaccharides) into disaccharides: Some glucometer and urine strips stix are used for testing glucose in blood and urine in diabetic and non-diabetic subjects. This means that existing glucometers and urine glucose strips are used to test only disaccharides in human products (blood and urine, etc).

The test with amylase also determines if a problem exists with the amylase secreted by diabetics in comparison with non-diabetics. Diabetic's amylase has prevented stain reaction between iodine and starch for approx. half an hour. In the same time, glucose resulted from starch conversion by amylase were higher in diabetic subjects in comparison with normal individuals.

The Dawn Phenomenon: A dawn phenomenon is the result of unconverted (slow converted), free blood starch (polysaccharides) in addition to tissue deposit of raw starch. The conversion of starch by amylase and other enzymes into sugar in diabetic patients and normal subjects takes place during the night. In diabetic patients this gives a significant rise in blood glucose levels during the early morning hours.

Test for Dawn Phenomenon: Patient take diabetes medication regularly and before bed time (insulin or tablets). Patient checks glucose levels immediately before taking medication (insulin or tablets), and 2 hours after medication before going to bed. Patient to take amylase and digestive enzymes (pancreatic-intestinal replacement therapy P.I.R.T) two hours after intake of medications and checking of glucose levels post medications. Patient checks the blood glucose levels early morning before treatment and breakfast (first thing in the morning).

Example of Results of the Test for Dawn Phenomenon: Blood glucose levels read no change or normal levels from the last reading obtained the night before (there will be no change or increase in blood glucose levels since last reading obtained within about 8 to 10 hours for a reading obtained two hours after treatment at bed time), that means there will be no dawn phenomenon. This will give a better control for diabetics and explains that blood and tissue starch are converted and utilized normally during the night by our new method of treatment and our new treatment by using pancreas-intestinal replacement therapy P.I.R.T. In addition this treatment can be used with insulin or oral hypoglycaemiants to control and treat diabetes or may replace insulin in treating diabetes after gradual withdrawal of insulin treatment in a way similar to withdrawal of steroids therapy. A treatment formula which contains sodium bicarbonate and P.I.R.T (pancreas intestine replacement therapy), is able to stop the so called dawn phenomenon, and in the same time managed to decrease glucose levels by 3 to 4 mmols during the night time (8 to 9 hours). Similar results were obtained by using insulin only before bedtime.

Another aspect of the invention provides a method of treating diabetes by administering amylase and/or replacing the exocrine pancreas (secretion) together with intestinal enzymes that converts disaccharides into monosaccharide by tablets or capsules.

Administration: The patient is first given sodium bicarbonate to drink for one or two days in order to leach starch from the body, then given amylase only or in combination with other enzymes (pancreatic and intestinal enzymes) in form of powder or capsule, e.g. 25 to 100 mg, three times a day, until the diabetes is controlled. This is long-term unless the diabetes is cured. Another aspect of the invention provides a method of testing, diagnosing or treating diabetes which aids conversion of polysaccharide to disaccharide. The methods also include giving amylase to convert polysaccharide into disaccharide.

Resistant starch that is not convert readily into disaccharide or monosaccharide, can be treated with sodium bicarbonate to leach out the starch, by breaking the molecular chains to make it easy for small amounts of amylase to convert polysaccharide to disaccharide. The present inventor therefore provides specific formulae capsules as hereinafter detailed; the capsules contents are meant to replace the exocrine pancreas together with intestinal enzymes (P.I.R.T pancreas, intestinal replacement therapy). The sodium bicarbonate can also be included in capsules.

Another aspect of the invention is a non-invasive method (fast, painless and convenient). In addition to accurate measurements of a real blood glucose could provide adequate control and greatly reduced the complications seen in these patients and consequently reduce health care cost.

Methods of Treatment. Treatment Test during day period: one embodiment of the present invention includes a method of Treatment includes: administering a sodium bicarbonate drink of about 200 to 500 mls of soda water containing between 100 to 200 mg of sodium bicarbonate (orally) to a patent, between one and three times a day. Administering the patent's normal insulin or oral diabetes medication as usual. The glucose levels were measured regularly at between 2 and 3 times per day. This method is repeated long-term unless the insulin or treatment is reduced or diabetes is cured. Comments: Sodium bicarbonate will leach starch out of tissues, capillaries and blood cells in the same time the starch molecule will be destabilized by the presence of sodium bicarbonate into small molecules with weak bonds and this will make it easy for amylase to convert into disaccharides which will result on a rise on blood sugar level by approximately 5 to 10 mmols when measured with normal glucometer. The administration of insulin or hypoglaycemiant medications (tablets) will reduce sugar levels to normal but will require high dose of insulin or tablets this normally occur during rest (no exercise). If the patient is to exercise even the slightest exercise, his or her blood sugar will decrease dramatically and can go into hypoglycemia very quickly. This method will reduce HbA1C levels almost in the same day by about 2 to 3%, and this will gradual decrease until returning to near normal or normal within 4 to 5 days.

Tables 1 and 2 represents readings of glucose levels and insulin doses on a Type I diabetic patient. Patient Name: M, A; Sex: Male; Age: 15 Years; Diabetes type: Patient with Type I diabetes; and Treatment: Insulin. The patient started using the first method of treatment (day and night), then changed to the 3^{rd} method after 2 weeks of treatment, carried n with it continuously.

TABLE 1

| | Mon | Tues | Wed | Thu | Fri | Sat | Sun |
|---|---|---|---|---|---|---|---|
| Pre Breakfast BG | 10.1 | 8.3 | 13.5 | 5.9 | 22.4 | 19.3 | 11.6 |
| Morning insulin | 30 | 30 | 30 | 30 | 30 | 30 | 30 |
| Site given | | | | | | | |
| Pre lunch BG | 9.3 | 7.2 | 5.1 | 12.2 | 11.1 | 12.3 | 14.6 |
| Lunchtime insulin | | | | | | | |
| Site given | | | | | | | |
| Pre tea BG | 23.1 | 26.2 | 18.4 | 22.2 | 19.5 | 19.9 | 17.4 |
| Pre tea insulin | 30 | 30 | 30 | 30 | 30 | 30 | 30 |
| Site given | | | | | | | |
| Pre bed time snack BG | | | | | | | |
| Pre bed time insulin | | | | | | | |
| Site given | | | | | | | |
| During night BG & | | | | | | | |
| Time | | | | | | | |
| Other BG test times | | | | | | | |
| Ketones/time tested | | | | | | | |
| Additional comments | | | | | | | |
| and information | | | | | | | |

TABLE 2

| | Mon | Tues | Wed | Thu | Fri | Sat | Sun |
|---|---|---|---|---|---|---|---|
| Pre Breakfast BG | 14.2 | 13.5 | 5.7 | 11.2 | 12.3 | 10.0 | 9.1 |
| Morning insulin | 10 | 10 | no | 10 | 10 | 5 | 10 |
| Site given | | | | | | | |
| Pre lunch BG | 8.3 | 9.2 | 5.4 | 5.1 | 9.2 | 4.5 | 8.4 |
| Lunchtime insulin | | | | | | | |
| Site given | | | | | | | |
| Pre tea BG | 15.6 | 18.4 | 14.6 | 19.6 | 16.7 | 21.2 | 15.4 |
| Pre tea insulin | | | | | | | |
| Site given | | | | | | | |
| Pre bed time snack BG | 22.6 | 14.4 | 26.2 | 18.2 | 16.7 | 23.4 | 16.8 |
| Pre bed time insulin | 20 | 20 | 30 | 20 | 20 | 30 | 20 |
| Site given | | | | | | | |
| During night BG & | | | | | | | |
| Time | | | | | | | |
| Other BG test times | | | | | | | |
| Ketones/time tested | | | | | | | |
| Additional comments | | | | | | | |
| and information | | | | | | | |

Table 1 shows glucose readings and insulin doses on a Type I diabetic patient one week before starting treatment. The patient was on 30 iu of insulin twice a day (60 iu/day) regardless of any reading of his glucose levels. Table 2 represents a table showing blood glucose levels and treatment readings for the same patient in Table 1. After 4 weeks of continuous treatment with P.I.R.T (treatment method number three), his insulin does was reduced to 10 iu in the morning and 20 iu in the evening (30 iu per day).

In another example: Table 3 and 4 represents a table show readings of glucose levels and insulin doses on a Type I diabetic patient. Patient Name: A, B; Sex: Male; Age: 46 Years; Diabetes type: Patient with type II diabetes; and Treatment: Tablets. The patient used initially the first method of treatment for (during day and night) two weeks then changed to the 3rd method of treatment by using only Pancreas Intestine Replacement Therapy (P.I.R.T), with intermittent use of sodium bicarbonate once every one or two weeks. The initial treatment and readings before seen in Tables 3 and after P.I.R.T treatment as seen in Tables 4.

TABLE 3

| | Mon | Tues | Wed | Thu | Fri | Sat | Sun |
|---|---|---|---|---|---|---|---|
| Pre Breakfast BG | 8.2 | 10.5 | 7.3 | 12.5 | 8.6 | 10.0 | 9.5 |
| Morning insulin | tab | tab | tab | tab | tab | tab | tab |
| Site given | | | | | | | |
| Pre lunch BG | 10.2 | 14.5 | 11.5 | 7.7 | 18.2 | 22.1 | 16.1 |
| Lunchtime insulin | tab | tab | tab | tab | tab | tab | tab |
| Site given | | | | | | | |
| Pre tea BG | | | | | | | |
| Pre tea insulin | | | | | | | |
| Site given | | | | | | | |
| Pre bed time snack BG | 10.1 | 12.4 | 9.8 | 4.5 | 15.7 | 13.9 | 14.3 |
| Pre bed time insulin | tab | tab | tab | tab | tab | tab | tab |
| Site given | | | | | | | |
| During night BG & | | | | | | | |
| Time | | | | | | | |
| Other BG test times | | | | | | | |
| Ketones/time tested | | | | | | | |
| Additional comments | | | | | | | |
| and information | | | | | | | |

TABLE 4

| | Mon | Tues | Wed | Thu | Fri | Sat | Sun |
|---|---|---|---|---|---|---|---|
| Pre Breakfast BG | 7.2 | 6.5 | 8.3 | 5.5 | 6.6 | 7.0 | 6.5 |
| Morning insulin | caps | caps | caps | caps | caps | caps | caps |
| Site given | | | | | | | |
| Pre lunch BG | 6.2 | 9.5 | 7.5 | 7.7 | 8.2 | 8.1 | 6.1 |
| Lunchtime insulin | caps | caps | caps | caps | caps | caps | caps |
| Site given | | | | | | | |
| Pre tea BG | | | | | | | |
| Pre tea insulin | | | | | | | |
| Site given | | | | | | | |
| Pre bed time snack BG | 7.1 | 6.4 | 7.2 | 4.5 | 5.7 | 7.9 | 5.3 |
| Pre bed time insulin | caps | caps | caps | caps | caps | caps | caps |
| Site given | | | | | | | |
| During night BG & | | | | | | | |
| Time | | | | | | | |
| Other BG test times | | | | | | | |
| Ketones/time tested | | | | | | | |
| Additional comments | | | | | | | |
| and information | | | | | | | |

Table 3 shows reading of glucose levels and insulin doses on a Type II diabetic patient one week before starting treatment. Patient on oral hypoglycemic agents for type II diabetes. Readings on this table obtained before commencing the new treatment with Pancreas Intestinal Replacement Therapy (P.I.R.T).

Table 4 shows blood glucose and treatment readings for the same patient in Table 3. After five months of continuous treatment using Pancreas Intestinal Replacement Therapy Treatment (enzyme replacement therapy treatment P.I.R.T). The patient is almost cured from diabetes.

Another embodiment of the present invention includes a method of treatment. The method of treatment includes administering to the patent sodium bicarbonate and regular treatment for about three days. Followed by administering to the patent sodium bicarbonate drink between 200 to 500 mls of soda water containing between about 100 to 200 mg of sodium bicarbonate (orally), about two to 3 times a day for 3 days. Administering to the patent normal insulin or oral diabetes medication as usual. Measuring the glucose levels about 2 to 3 times per day. Followed by pancreatic-intestine enzymes replacement therapy in addition to usual treatment continuously. Continue the treatment until the method unless the insulin or treatment does is reduced or diabetes is cured. Comments: The method contains an initial sodium bicarbonate treatment for about 3 days to leach starch out of tissues and cells (starch clearance). The administration of insulin or oral treatment will reduce sugar levels to normal but will require high dose of insulin or tablets during the first 2 to 3 days, and this is normally occur during the rest (no exercise) period. If the patient is to exercise even the slightest exercise, his or her blood sugar will decrease dramatically and can go into hypoglycemia very quickly. After 3 to 4 days the sugar levels will decrease gradually. Treatment dose of pancreatic-intestine enzymes replacement therapy has to be increased according to meals. This method will reduce HbA1C levels almost in the same day by 2 to 3%, but after 4 to 5 days HbA1C levels will return to near normal or normal.

Another embodiment of the present invention includes a method of treatment. The method of treatment includes administrating pancreatic-intestine enzymes replacement therapy to a patent.

Administering insulin or oral diabetes medication to the patent and continuing the treatment with pancreatic-intestine enzymes replacement therapy (P.I.R.T). Repeat the method unless the insulin or treatment does is reduced or diabetes is cured. Comments: This method will control blood sugar gradually. It will require high insulin doses at the beginning and blood sugar levels some time can be normal or slightly high during rest (no exercise) period. If the patient is to exercise even the slightest exercise, his or her blood sugar will decrease but gradually and within reasonable reading for example between 9 to 10 or 10 to 13 mmols. With heavy exercise, sugar levels can easily return to normal and stays normal for a long time despite consumption of sugar, sweets and starchy meals. HbA1C levels will be reduced to near normal or normal almost on the same day or within 2 or 3 to 5 days.

Another embodiment of the present invention includes a method of treatment during night periods: The method includes administrating sodium bicarbonate drink about 200 to 500 mls of soda water containing between about 100 to 200 mg of sodium bicarbonate (orally), two or more times a day. Administering an insulin or oral diabetes medication to the patent and measuring the glucose levels regularly two or more times per day. Repeat the method unless the insulin or treatment is reduced or diabetes is cured. Comments: Sodium bicarbonate will leach starch out of tissues, capillaries and blood cells in the same time the starch molecule will be destablized by the presence of sodium bicarbonate into small molecules with weak bonds and this will make it easy for amylase to convert into disaccharides which will result on a rise on blood sugar level by approximately 5 to 10 mmols when measured with normal glucometer. The administration of insulin or hypoglaycemiant medications (tablets) will reduce sugar levels to normal but will require high dose of insulin or tablets this normally occur during rest (no exercise). If the patient is to exercise even the slightest exercise, his or her blood sugar will decrease dramatically and can go into hypoglycemia quickly. Glucose level will be reduced all-night through, and in the morning sugar levels will be within normal or slightly high by 2 to 3 mmol in the first few days, then morning sugar levels will return to near normal or normal within a period of 2 to 3 weeks. This method will reduce HbA1C levels almost in the same day by 2 to 3%, and this will gradual decrease until returning to near normal or normal within 4 to 5 days.

The HbA1C test is a hemoglobin specific test for diabetes. The test indicates how well diabetes is being controlled in a patient. It is usually measured every two to three months, some times 2-3 weeks. The results for a normal non-diabetic person are about 4 to 6% of HbA1C in red cells. In patents with uncontrolled diabetic, the result is greater than 6%, e.g. 7 to 16%.

Yet another embodiment of the present invention includes a method of Treatment during night period: The method of treatment includes administering to the patent sodium bicarbonate and regular treatment for about three days. The sodium bicarbonate drink about 200 to 500 mls of soda water containing between about 100 to 200 mg of sodium bicarbonate (orally), every two or more times a day. Measuring the glucose levels regularly two or more times per day. Follow by discontinued admonition of sodium bicarbonate to the patent. Administering to the patent pancreatic-intestine enzymes replacement therapy. Repeat the method unless the insulin or treatment is reduced or diabetes is cured. Comments: This method uses initially sodium bicarbonate administered for 3 days to leach starch out of tissues and cells (starch clearance). The administration of insulin or oral treatment will reduce sugar levels to normal but will require high dose of insulin or tablets during the first 2 to 3 days, and this is normally occur during rest (no exercise) periods. If the patient is to exercise even the slightest exercise, the blood sugar will decrease dramatically and can go into hypoglycemia very quickly. After 3 to 4 days sugar levels will decrease gradually. Treatment dose of pancreatic-intestine enzymes replacement therapy has to be increased according to meals. Morning sugar levels will be near normal or slightly high during the first 5 to 7 days, and then become static (meaning same night blood sugar reading taken before bed will be approximately the same reading in the following morning). The blood sugar levels will start to decrease gradually by 2 to 4 mmol before bed and early morning. For example: The blood sugar levels after one hour from insulin dose recorded as 10 mmols, 3 hours later this will be 8 mmols and early morning will read 6 to 7 mmols. This method will reduce HbA1C levels almost in the same day by 2 to 3%, but after 4 to 5 days HbA1C levels will return to near normal or normal.

Table 5 shows readings of glucose levels and insulin doses on a Type I diabetic patient one week before starting treatment with sodium bicarbonate (ingestion of sodium bicarbonate every day). Patient Name: M, Z; Sex: Male; Age: 13 Years. Diabetes type: Patient with Type I diabetes; and Treatment: Insulin. During week days the patient is active while at school and less active during the weekends. The blood glucose levels were high during weekends and his insulin requirements during the same weekends were also high compared to readings taken during weekdays in the same week.

Table 6 shows blood glucose and treatment readings for the same patient in Table 5. Patient started school holiday together with the start of new treatment method number one (daily intake of sodium bicarbonate). During this first week of school holiday, patient was less active in comparison with previous normal school week. Note the gradual slight change on his glucose readings during week days and weekends in comparison with previous week.

TABLE 5

| | Mon | Tues | Wed | Thu | Fri | Sat | Sun |
|---|---|---|---|---|---|---|---|
| Pre Breakfast BG | 8.3 | 4.8 | 17.8 | 3.7 | 4.8 | 15.5 | 14.6 |
| Morning insulin | 36 | 36 | 38 | 36 | 34 | 38 | 38 |
| Site given | R.L | L.L | R.L | L.L | R.L | L.L | R.L |
| Pre lunch BG | | | 17.8 | 4.9 | | 10.3 | 14.2 |
| Lunchtime insulin | | | | | | | |
| Site given | | | | | | | |
| Pre tea BG | 11.8 | 15.4 | 22.1 | 7.4 | | 10.1 | 12.8 |
| Pre tea insulin | 7 | 8 | 9 | 7 | | 8 | 8 |
| Site given | | | | | | | |
| Pre bed time snack BG | 11.9 | 7.4 | 5.4 | 6.0 | 6.8 | 5.5 | 13.5 |
| Pre bed time insulin | 9 | 9 | 9 | 8 | 7 | 8 | 9 |
| Site given | | | | | | | |
| During night BG & | | | | | | | |
| Time | | | | | | | |
| Other BG test times | | | | | | | |
| Ketones/time tested | | | | | | | |
| Additional comments | | | | | | | |
| and information | | | | | | | |

TABLE 6

| | Mon | Tues | Wed | Thu | Fri | Sat | Sun |
|---|---|---|---|---|---|---|---|
| Pre Breakfast BG | 17.6 | 6.7 | 4.3 | 4.6 | 7.1 | 10.0 | 13.1 |
| Morning insulin | 36 | 36 | 36 | 34 | 36 | 38 | 40 |
| Site given | L.L | L.L | R.L | L.L | R.L | L.L | R.L |
| Pre lunch BG | 14.8 | 3.8 | | | 10.3 | 19.8 | 19.2 |
| Lunchtime insulin | | | | | | | |
| Site given | | | | | | | |
| Pre tea BG | 10.9 | 7.2 | 14.6 | 9.7 | 9.5 | 7.1 | 15.4 |
| Pre tea insulin | 7 | 7 | 8 | 6 | | 7 | 8 |
| Site given | | | | | | | |
| Pre bed time snack | | 4.3 | 4.1 | 5.6 | 6.1 | 10.6 | 11.7 |
| BG | | | | | | | |
| Pre bed time insulin | 9 | 8 | 7 | 6 | 7 | 9 | 9 |
| Site given | | | | | | | |
| During night BG & | | | | | | | |
| Time | | | | | | | |
| Other BG test times | | | | | | | |
| Ketones/time tested | | | | | | | |
| Additional | | | 3 | | | | |
| comments and | | | TIME | | | | |
| information | | | HYPO | | | | |

Yet another embodiment of the present invention includes a method of treatment during night period. The method of treatment includes administering to the patent pancreatic-intestine enzymes replacement therapy and by administering to the patent normal insulin or oral diabetes medication. This is a long-term method of treatment unless the insulin or treatment is reduced or diabetes is cured. Comments: This method will control blood sugar gradually. It will require high insulin doses at the beginning and blood sugar levels some time can be normal or slightly high during rest (no exercise) period. If the patient is to exercise even the slightest exercise, the blood sugar will decrease but gradually and within reasonable reading for example between 9 to 10 or 10 to 13 mmols. With heavy exercise, sugar levels can easily return to normal and stays normal for a long time despite consumption of sugar, sweets and starchy meals. Blood sugar levels measured before bed will be within normal range or slightly high (10 to 11 mmols) initially, that is to say during the first 2 to 3 weeks, then decrease gradually to become near normal or normal. Blood sugar levels obtained through this method will maintain near normal or normal readings for a long time despite consumption of sugar, sweets and starchy meals. Some people has become insulin free through this method, and some other patients are cured. HbA1C levels will be reduced to near normal or normal almost on the same day or within 2 to 5 days.

One example of a pharmaceutical formulation includes a capsule containing:
Amylase 100 mg;
Maltase 10 mg;
Sucarase 10 mg;
Lactase 10 mg; and
Cellulase 10 mg.

Another example of a pharmaceutical formulation includes a capsule containing:
Amylase 50 mg;
Lipase 80 mg;
Protease 100 mg;
Maltase 20 mg;
Lactase 20 mg;
Cellulase 20 mg; and
Sodium bicarbonate 0.5 mg.

Another example of a pharmaceutical formulation includes a capsule containing:
Amylase 100 mg;
Maltase 10 mg;
Sucarase 10 mg;
Lactase 10 mg;
Cellulase 10 mg;
Invertase w/w;
Alpha galactosidase (providing 1000 AGSU);
Amyloglucosidase 26 mg;
Protease 40 mg;
Papain 26 mg;
Bromelain 26 mg; and
Lipase 26 mg.
   Encapsulated with these natural ingredients: Dicalcium phosphate, magnesium stearate.

Another example of a pharmaceutical formulation includes a capsule containing:
Amylase 50 mg;
Maltase 10 mg;
Sucarase 10 mg;
Lactase 10 mg;
Cellulase 10 mg;
Invertase w/w;
Alpha galactosidase (providing 1000 AGSU);
Amyloglucosidase 26 mg;
Protease 40 mg;
Papain 26 mg;
Bromelain 26 mg; and
Lipase 26 mg.
   Encapsulated with these natural ingredients: Dicalcium phosphate, magnesium stearate.

Another example of a pharmaceutical formulation includes a capsule containing:
Amylase 50 mg;
Glucoamylase 4.5 AGU;
Maltase 10 mg;
Sucarase 10 mg;
Lactase 10 mg;
Cellulase 10 mg;
Invertase 100 SU;
Alpha galactosidase (providing 1000 AGSU);
Amyloglucosidase 26 mg;
Protease 40 mg;
Papain 26 mg;
Bromelain 26 mg;
Lipase 26 mg; and
Sodium bicarbonate 1.25 g.
   Encapsulated with these natural ingredients: Dicalcium phosphate, magnesium stearate.

Another example of a pharmaceutical formulation includes a capsule containing:
Amylase 25 mg;
Maltase 10 mg;
Sucarase 10 mg;
Lactase 10 mg;
Cellulase 10 mg;
Alpha galactosidase (providing 1000 AGSU);
Amyloglucosidase 26 mg;
Protease 40 mg;
Papain 26 mg;
Bromelain 26 mg;
Lipase 26 mg; and
Sodium bicarbonate 2 g.
   Encapsulated with these natural ingredients: Dicalcium phosphate, magnesium stearate.

Another example of a pharmaceutical formulation includes a capsule containing:
Amylase 25 mg;
Glucoamylase 4.5 AGU;
Maltase 10 mg;
Malt diastase 270 DP;
Sucarase 10 mg;
Lactase 10 mg;
Cellulase 10 mg;
Invertase 100 SU;
Alpha galactosidase (providing 1000 AGSU);
Amyloglucosidase 26 mg;
Protease 40 mg;
Papain 26 mg;
Pectinase (with phytase) 75 ENDQ/PGU;
Bromelain 26 mg;
Lipase 26 mg; and
Sodium bicarbonate 500 mg.
   Encapsulated with these natural ingredients: Dicalcium phosphate, magnesium stearate.

The pharmaceutical formulation may includes capsules, tablets, medicinal syrup with different flavors, dissolvable tablets, and effervescent tablets containing sodium bicarbonate. Different strength can be provided. Another example of a pharmaceutical formulation includes capsules, powder, effervescent powder, effervescent tablets containing sodium bicarbonate plus amylase (plant, animal or human origin, micro or macroamylase). different strength can be provided. weight will be in mg or gram. Additionally, Substances of plant origin or animal source substances can also be used. Yet another example of a pharmaceutical formulation includes capsules, tablets, medicinal syrup with different flavors, dissolvable tablets, and effervescent tablets, skin patches, drops, I.V preparations (e.g., infusion, i.v, intramuscular or subcutaneous injections), suppositories, creams, solutions, powders, sprays, or wound dressings. The formulation containes amylase of plant origin, animal origin or human origin: microamylase and macroamylase. Additionally, formulations of different strength can be provided.

The present invention may be used in a method of treatment and prophylaxis for high Cholesterol levels: The method includes administering about 300 mg/kg/body weight sodium bicarbonate to a patent. Measure the cholesterol levels of the patent. Whereby the method help prevent deposition of starch in blood, arteries and tissues, and in the same time lower cholesterol levels.

The pharmaceutical formulation may be in the form of capsules, tablets, medicinal syrup with different flavors, dissolvable tablets, and effervescent tablets or powder, skin patches, drops, i.v preparations (infusion, i.v, intramuscular or subcutaneous injections), suppositories, vaginal tablets, vaginal passerines, vaginal ointment, vaginal cream, other preparations (solution, spray, powder, ointment, cream and wound dressing tissues containing powder or solution) for topical use and dressing for skin wounds and ulcers.

Example: Sodium Bicarbonate and polysaccharides: Demonstrate that polysaccharides are present in blood and tissues as a slow release energy source, that can be used to enhance exercise performance when treated with sodium bicarbonate and the role of sodium bicarbonate, as an ergogenic aid to enhance exercise performance by destabilization of body polysaccharides, so it can be used as an extra energy during exercise.

Example: Fifteen healthy male volunteers of different age groups varies between 20 to 32 years and body weight between 65 to 70 kg, male subjects, were put for exercise test under three conditions: following ingestion of 300 mg sodium bicarbonate per kg of body mass (i); following ingestion of a placebo (100 mg sodium chloride isotonic solution per kg of body mass) (ii); and following ingestion of neither (iii). A double-blind protocol was used between the (i) and (ii) trials. All volunteers had a starchy meal before going to bed the night before exercise. Each condition was repeated so that the volunteers underwent treadmill exercise for six times. 100 min before commencing treadmill exercise was allowed after ingesting substances in (i), (ii) and (iii). The volunteers exercised until fatigue.

Results: Fourteen of the volunteers completed all the tests. The volunteer's average times for trials (i), (ii) and (iii) were 4.01, 4.25 and 4.36.0 s, respectively. The data were analyzed using a two-way ANOVA with replicates and Tukey tests. This revealed a difference between trial (i) and trials (ii) and (iii) (P<0.05), but no difference between trials (ii) and (iii). Conclusion: The findings, therefore, indicated that sodium bicarbonate can have an ergogenic effect upon exercise.

Amylase quality and function test in diabetes and health subjects. Materials and Methods. Amylase test in normal and diabetic subjects Tests (I) and (II). (I) First Test: Amylase Polysaccharides (Starch) Test. Materials. The materials include: Specimen pots with lid; Small piece of breads 1 to 2 mg in weight or flour; isotonic solution of normal saline 25 mls; amylase normal healthy human saliva 25 mls; amylase diabetic 30 Type I human saliva 25 mls; amylase diabetic Type II human saliva 25 mls; iodine tincture; sodium bicarbonate powder; blood glucose strips and glucometer; urine glucose strips; and watch for timing. Furthermore, pancreatic amylase can also be used instead of salivary amylase.

The Iodine Test Method included: Cleaning the mouth thoroughly by using toothbrush and rinsing mouth with water thoroughly. Saliva was collected and an amount of 25 mls of normal healthy subject saliva was placed in a specimen pot (A). Type I diabetic patient saliva, 25 mls, was placed in specimen pot (B); Put 25 mls of Type II diabetic patient saliva in a specimen pot (C); A small piece of white bread 1 to 2 mg was placed in each specimen pots A, B, and C. Time is then be recorded and additional iodine drops (e.g., 5 drops) are added to each specimen pot immediately. Record any change in color. Repeated the iodine test again every 5 minutes for one and a half hours and record any change in color in each specimen pot; Repeated the iodine test every one-hour after the first half an hour and for two (2) hours and record any change in color; Repeated the iodine test, in each specimen pot 4 hourly for 24 hours and record any change in color.

Results: The results form the Iodine Test include: In specimen pot (A), there was immediate color reaction when iodine is added. The piece of bread was stained with dark blue color (blue color noted also, on some parts of the bread and the solution). In specimen pot (B), there were no immediate changes in color for the bread and the solution. The iodine yellow brown color persisted without staining the bread, and the diabetic Type I saliva (e.g., amylase) may be visualized, preventing reaction between iodine and starch (e.g., polysacharides). The polysaccharides were shielded from iodine stain reaction. No color reaction between iodine and bread was recorded for approximately 20 to 30 minutes. After approximately 20 to 30 minutes a stain reaction developed gradually and slowly.

In specimen pot (C), there were no immediate changes in color of the bread or the solution. The iodine yellow brown color persisted without stain reaction with polysaccharides or the solution and diabetic Type II saliva (e.g., amylase) may be visualized, prevented immediate stain reaction between starch and polysaccharides. No color reaction between iodine and bread was recorded for approximately 10 to 15 minutes. After approximately 10 to 15 minutes a stain reaction developed gradually and slowly. Final iodine test carried out after twenty-four hours recorded no color reaction between iodine and bread and the iodine yellow brown color remained unchanged for approximately one hour then the solution and the bread become stain free again.

The method of testing the glucose levels include: checking glucose levels in each specimen pot every half an hour for the first two hours, then every four hours for twenty-four hours (e.g., measurement to be done using normal glucometer and urine strip test for glucose). Checking the final glucose levels in each specimen after twenty-four hours and record the results.

Results: Results of the of the glucose test includes: Glucose test results were obtained after twenty-four hours from each specimen pot and the result were as follows: In specimen pot (A), glucometer tested positive for glucose 7 mmols, and urine strip test was positive for glucose and displayed only one cross (+). In specimen pot (B), glucometer tested positive for glucose 16 mmols, and urine strip test tested positive for glucose and displayed two crosses (++). In specimen pot (B), glucometer tested positive for glucose 10 mmols, and urine strip test tested positive for glucose and displayed one cross (+).

Interpretation of results. The addition of amylase to starch (saliva amylase or plant, etc), this will convert starch (polysaccharides) into disaccharides and following this fact the following conclusions were drawn: In the amylase starch saliva experiment tested for disaccharides, which resulted from starch conversion by amylase. Same glucometer and urine strips are used for testing glucose in blood and urine in diabetic and non-diabetic subjects. This means that existing glucometers and urine glucose strips are used to test only disaccharides in human products (blood and urine, etc). The test with amylase also concludes that there is a problem with the amylase secreted by diabetics in comparison with non-diabetics. Diabetic's amylase has prevented stain reaction between iodine and starch for approx. half an hour. In the same time, glucose resulted from starch conversion by amylase were higher in diabetic subjects in comparison with normal individuals.

Amylase reacts with Polysaccharides to converts the polysaccharides into disaccharides. Saliva amylase polysaccharides test (e.g., diabetics saliva and normal healthy subjects' saliva) results showed saliva malfunction reaction with starch in diabetic subjects. The following test demonstrates that saliva amylase, pancreatic amylase and amylase of other origin for example plants, animal etc has strong important reactions with fast immediate effects on disaccharides. This test was not known and never been demonstrated the inventor's way before.

A second Test: Amylase Disaccharides (sucrose) Test. This test demonstrates quality of amylase and proves that amylase is needed for breakdown of disaccharides (e.g., amylase react with disaccharides like sucrose).

Materials: The materials for the Amylase Disaccharides (sucrose) Test include: Specimen pots with lid×10; Table sugar (sucrose) teaspoon full (5 mls/5 mg)×5; Isotonic solution of normal saline 25 mls×4; Amylase normal healthy human saliva 25 mls×2; Amylase diabetic Types I, human saliva 25 mls×2; Amylase diabetic Types II, human saliva 25 mls×2; Amylase exogenous plant origin (100 mg powder); Blood glucose strips and glucometer; Urine glucose strips; and a Watch for timing. Furthermore, Pancreatic amylase can also be used instead of salivary Amylase.

A second test method. Amylase disaccharides (sucrose) test method. Clean the mouth by using toothbrush and rinse mouth with water thoroughly. Start collecting saliva; Put an amount of about 25 mls. of normal healthy subject saliva in a specimen pot (A) and (B); Put 25 mls. of Type I diabetic patient saliva in specimen pot (C) and (D); Put 25 mls of Type II diabetic patients saliva in a specimen pot (E) and (F); Put 100 mg of exogenous plant amylase in specimen pot (G) and (H); Add 25 mls of normal saline isotonic solution in specimen pot (G) and (H) and shake well; Put 25 mls of normal saline isotonic solution in specimen pot (I) and (J); Put a full teaspoon 5 mls/5 mg table sugar (sucrose) in specimen pots A, C, E, G and I. Time is the recorded. Check sugar levels in each specimen pot using normal glucometer with suction strips, every 5 minutes for half an hour (in another embodiments the time may be one hour) then hourly for twenty-four hours; and in the same time; Check sugar levels in each specimen pot using urine glucose strips (example, diastix by bayer), every five (5) minutes for half an hour (in another embodiments the time may be one (1) hour) then hourly for twenty-four hours; and record sugar levels in each specimen pot.

Second Test Results. Amylase Disaccharides (sucrose) Test Results. After 5 minutes results: In specimen pot (A), (normal healthy subjects saliva amylase and Sucrose only) there was immediate reaction between sucrose and amylase and glucometer reading was low (below 1.1 mmol). Urine glucose strip (diastix) recorded very mild discoloration. In specimen pot (B), (Normal Healthy subjects saliva amylase only), there was no reaction and the glucometer reading was error two (2) (Technical error and requesting to check strips or battery). Urine glucose strips showed no change in color. In specimen pot (C), (Type I diabetic saliva amylase and sucrose) there was no reaction and the glucometer reading was error two (2) (Technical error and requesting to check strips or battery). Urine glucose strips showed no change in color. In specimen pot (D), (Type I diabetic saliva amylase only), there was no reaction and the glucometer reading was error two (2) (Technical error and requesting to check strips or battery). Urine glucose strips showed no change in color. In specimen pot (E), (Type II diabetic saliva amylase and Sucrose) there was no reaction and the glucometer reading was error two (2) (Technical error and requesting to check strips or battery). Urine glucose strips showed no change in color. In specimen pot (F), (Type II diabetic saliva amylase only), there was no reaction and the glucometer reading was error two (2) (e.g., technical error and requesting to check strips or battery). Urine glucose strips showed no change in color. In specimen pot (G), (e.g., 100 mg exogenous plant amylase and normal saline and sucrose), there was no reaction and the glucometer reading was error two (2) (Technical error and requesting to check strips or battery). Urine glucose strips showed no change in color. In specimen pot (H) (100 mg exogenous plant amylase and normal saline), there was no reaction and the glucometer reading was error two (2) (Technical error and requesting to check strips or battery). Urine glucose strips showed no change in color. In specimen pot (I) (e.g., normal saline and sucrose only), there was no reaction and the glucometer reading was error two (2) (e.g., technical error and requesting to check strips or battery). Urine glucose strips showed no change in color. In specimen pot (J) (e.g., normal saline only), there was no reaction and the glucometer reading was error two (2) (e.g., technical error and requesting to check strips or battery). Urine glucose strips showed no change in color. Same results (with no changes) were obtained every five (5) minutes for the first half an hour. After the first Half an hour, diabetes Type I and II saliva and sucrose started to give glucometer reading of low (below 1.1 mmols), and urinary strips showed mild color changes.

Normal healthy amylase and sucrose sample together with exogenous 100 mg plant amylase and sucrose sample started to read higher levels of glucose and by eight (8) hours glucose reading in these samples was around 14 mmols.

Sucrose and normal saline or water (e.g., table sugar dissolved in water) sample (Specimen pot (I) recorded no reaction for up to twenty-four (24) hours, the glucometer reading was always error two (2) (Technical error and requesting to check strips or battery). Urine glucose strips showed no change in color for 24 hours.

Comments: Table sugar (disaccharide) can be recorded with glucometer and urine glucose strip in the presence of amylase only. Diabetes amylase (diabetes saliva amylase) was unable to make the table sugar recordable with glucometer and urine glucose strip for the first half-hour of the contact between diabetes saliva and sucrose. glucometer reading and urine glucose strip reading for specimen pot (I), (e.g., sucrose and normal saline), was similar to that of diabetes saliva amylase reaction with glucose for the first half-hour (no reaction).

Summery: Amylase reacts with both polysaccharides and disaccharides and makes both of them recordable (e.g., readable) by the normal glucometers and urinary glucose strips. Normal healthy subjects amylase action on disaccharides is almost immediate (e.g., fast, rapid reaction), but diabetes amylase reaction with disaccharides is delayed for approximately half an hour or more initially. Conclusion: In diabetes, disaccharides and polysaccharides do not get processed and prepared by saliva Amylase and the same problem probably happens, with pancreatic amylase.

Raw unprocessed disaccharides and polysaccharides get absorbed into the blood and circulate in the blood until they are gradually or rapidly processed by the amylases in the blood. Approximately 50% of the ingested disaccharides and polysaccharides probably get processed normally in the digestive system to maintain blood sugar levels of 4 to 6 mmols. The other remaining 50% of the ingested disaccharides and polysaccharides get absorbed directly into the blood stream as a raw material. These raw disaccharides and polysaccharides are then processed in the blood stream by the blood amylases, which will elevate sugar levels. This process would normally happen in the mouth and rest of the digestive system, however this occurs in the blood instead. This is one explination of the high sugar levels associated with diabetes.

For more accurate test results both tests may be applied together: Amylase polysaccharides (starch) test (iodine version). Amylase disaccharides (sucrose) test. Both tests should be equal in time units functions, that is to say time results for the amylase iodine starch test should be equal to that of Amylase sucrose test. (For example in normal person is between about 0 and 3 minutes, however in Type I diabetes is 30 minutes before you can obtain any normal like reaction).

Amylase, disaccharides, polysaccharides quality and function tests: new tests and new testing machines (testing devices or apparatus) to determine quality and functions of amylase (salivary or pancreatic or exogenous of animal or plant origin): Until now tests on amylases are only developed to measure the levels of amylase in the blood. The present invention includes a tests by which he can determine the quality of amylase especially salivary amylase and pancreatic amylases. The present invention will help screen and diagnoses in addition to follow up and monitor people and animals with amylase related diseases such as diseases of the pancreas including diabetes, and salivary glands, or to determine whether the intestines have been damaged.

Summary of Experimental Results: Numerous studies have investigated the use of oral or intravenous NaHCO₃ as an ergogenic aid, but there appears to be a lack of agreement among studies concerning the effectiveness of NaHCO₃ as an ergogenic aid. In fact very little evidence exists suggesting that NaHCO₃ can increase work capacity or power output. Several reports indicate that NaHCO₃ can delay the onset of fatigue, but the exercise protocol employed and the dosage of NaHCO₃ utilized appear to have a considerable influence on the conclusions drawn from these studies.

During exhaustive exercise lasting 1 to 7 minutes, NaHCO₃ loading has been shown to delay the onset of fatigue. However, a single bout of supramaximal exercise lasting 1 minute or less is apparently too short in duration to benefit from enhanced buffer concentration. In contrast, when exhaustion occurs beyond about 7 minutes of exercise enhanced buffer concentration has not been shown to be effective. Thus, there appears to be a window of time in which NaHCO₃ enhances exercise performance this is likely due to the fact that exercise intensity and duration directly influence the energy system supporting the metabolic demands of exercise.

Accelerated utilization of glucose generated from destabilization of polysaccharides by insulin and amylase is apparently the mechanism by which NaHCO3 ingestion delays the onset of fatigue. During sprint-type exercise the metabolic demands of exercise are met primarily by immediate energy sources, and to a lesser extent anaerobic metabolism. The power output of these types of exercise protocols can not be sustained long enough for the intracellular environment to be affected by an enhanced extracellular buffer capacity. For similar reasons, there is no benefit of NaHCO3 ingestion during endurance exercise, since endurance exercise is primarily supported by oxidative metabolism with a smaller contribution of energy from polysaccharides aerobic glycolysis.

Sodium bicarbonate ingestion can increase maximal exercise tolerance and enhance athletic performance. Most notably Wilkes and co-workers reported a decrease in 800 m running time of nearly 3 seconds, which was estimated to be the equivalent of approximately 19 m in distance. Similarly, both Rupp et al. and Sutton et al reported that NaHCO₃ ingestion prolongs exercise duration at 95%. Additionally, Costill and co-workers, utilizing an interval protocol requiring four 1-minute maximal sprints at 125% with a 1-minute rest period between exercise bouts and a fifth interval to exhaustion, reported that NaHCO₃ ingestion prior to exercise increased exercise duration of the final bout by 42%.

In endurance events the fractional contribution of glycolysis to energy production is less than that for short-term maximal exercise. Consequently blood and muscle acidosis is not as pronounced. NaHCO₃ ingestion had no effect on time to exhaustion in subjects running at an intensity corresponding to a blood lactate of 4 mM, which is a commonly used marker of the onset of blood lactate accumulation. Subjects were able to sustain this intensity for approximately 30 minutes, indicating that this exercise intensity results in exhaustion well beyond the window of time discussed previously. Furthermore, in contrast to reports indicating that NaHCO₃ ingestion can prolong exhaustive exercise, few investigations have reported an increase in power output in subjects who ingested NaHCO₃.

Using either the Wingate Anaerobic Test (WAT), or a similar test requiring subjects to pedal against a fixed load for 30-60 s, mean power output is reportedly increased, but not peak power output. McKenzie et al. reported an increase in power output and time to exhaustion in a protocol similar to that of Costill and co-workers, described above. Additionally, Horswill and co-workers reported no significant increase in power output during a 2-minute maximal exercise bout on an isokinetic bicycle ergometer, attributing their findings to the low dosage of bicarbonate utilized in their study (100, 150, and 200 mg NaHCO₃/kg body weight), and suggesting that the relative dose of NaHCO₃ ingested is an important variable.

In general, studies that have utilized less than 300 mg NaHCO₃/kg body weight have reported no positive effect on exercise performance. Blood pH and bicarbonate ion concentration are generally higher in those studies where subjects ingested 300 mg NaHCO₃/kg body weight, compared to studies where subjects ingested 150-200 mg NaHCO₃/kg body weight. Direct comparison of these studies is difficult due to differences in blood sampling sites and use of different blood pools (i.e. venous or arterialized venous blood). Because efflux of lactate and hydrogen ions is directly related to extracellular pH, it is likely that a larger dose of NaHCO₃ results in a greater extracellular bicarbonate ion concentration that increases efflux of lactate and hydrogen ions and delays the onset of critically low intracellular pH. Studies employing a dosage of 200 mg NaHCO₃/kg body weight or less have shown an increase in resting bicarbonate ion concentration, but have not consistently shown an increase in exercise performance. However, more frequent reference has been made to subjects experiencing gastrointestinal discomfort by those investigators who have utilized 300 mg NaHCO₃/kg body weight.

Summary: It is clear that maximal exercise causes changes in blood and muscle acid-base balance that are related to fatigue and cessation of exercise. Recruitment of polysaccharides oxidative energy sources contributed in increasing and generation of metabolic acids which directly affect intracellular pH. A decrease in intracellular pH may contribute to the cessation of exercise by impairing generation of energy and inhibiting contractile function. Although intracellular bicarbonate ion concentration is unaffected by an increase in extracellular bicarbonate ion concentration, the onset of critically low intracellular pH can be delayed by increasing extracellular bicarbonate ion concentration. Lactate and hydrogen ion transport from the intracellular compartment to the extracellular environment is accomplished by a membrane bound lactate transporter, following a favorable pH gradient. An increase in extracellular bicarbonate ion concentration increases efflux of lactate and hydrogen ions.

Ingestion of NaHCO₃ has not been clearly shown to increase power output, but can apparently delay fatigue during short-term maximal exercise, and seems to be most effective during maximal exercise bouts resulting in fatigue in 1-7 minutes. Alternative exercise protocols and lower dosages of NaHCO₃ have not consistently demonstrated improved exercise performance. It is concluded that ingestion of NaHCO₃ can prolong high-intensity exercise, most likely due to destabilisation and semi-degradation of starch (free blood and tissue polysaccharides) by destabilising its molecular bonds (transforms the whole polysaccharide compound into an instable compound), and accordingly this makes it easy for the insulin to access the polysaccharides compound and act on the glucose by transforming it into energy, in the same time the amylase also will act to convert the rest of it into glucose and the glucose energy generated from the two processes will increase the function and activity of working muscle. In the same time, increased transport of lactate and H+ from the intracellular compartment of working muscle will interfere with muscle cells metabolism by delaying the onset of critical intramuscular pH that impairs metabolic and contractile function of working muscles.

Conclusion: Amylase is similar to insulin in function and sodium bicarbonate helps prepare polysaccharides for degradation and utilization for energy release. Insulin provides an immediate action to help provide energy, and Amylase provides slow action to maintain body energy. That is to say Insulin is used for immediate energy release and Amylase for slow energy release. Amylase and sodium bicarbonate supplements in addition to exercise where energy is in consumption and demand are important and should always be considered in normal healthy subjects and other subjects with starch related diseases including diabetes, so as to prevent complications such as obesity and other complications resulting from accumulation of polysaccharides (raw starch) in blood and tissues, and to utilize body energy resources properly.

Amylase Polysaccharides Testing Machine: A machine or device for testing quality and functions of amylases based on Amylase Polysaccharides Test color changing reaction per units of time (minutes or seconds) (Iodine Test). The machine will be as an infrared electrophotometer with a sample placement chamber. The chamber is to be fitted with a removable disposable sample container. Pre-tested standard type, standard amount sterile testing reagents of Polysaccharides will be available for use with the test to give good results.

The machine will be calibrated with normal standard values measured and obtained from the color changing time reaction that normally occurs between Polysaccharides and iodine in the presence of amylase staining Test. The given sample to be tested will be placed in a disposable sample container and placed in the testing sample chamber. Graphic results will also be obtained and printed out from the machine. This will show the correlation between time and color changes, and results will be compared with normal standard values for the test. A software program for this test will also be available.

Amylase Disaccharides Testing Machine: An apparatus, method, composition and device for testing quality and functions of amylases based on Amylase Disaccharides Test reaction per units of time (minutes or seconds). The principal and the main idea to be used in this machine will be similar to that used in the Amylase Disaccharide (sucrose) Test. The machine will be similar to a glucometer but slightly larger. It will be fitted with a sample placement chamber with a fixed sensor electrode in the base or sides, which will be connected to a disposable sample container (the idea of the disposable sample container is similar to glucometer testing strips). This will be used only once. It will be for either a continuous time reading or limited time reading. Pre-tested standard type, standard amount sterile testing reagent of disaccharides will be available for use with the test to give good results. Graphic results will also be obtainable and printable from the machine. This will show the correlation between time and reaction changes, and results will be compared with normal standard values for the test. A software program for this test will also be available.

The idea is similar to that described in the basic (e.g., Amylase Disaccharides Test by using Sucrose, although other Disaccharides can also be used for this test). New Testing machines (Testing devices or apparatus) to determine quality of polysaccharides or disaccharides, G.M Food, for individual use, hospital use, industry use, food production use and others.

Currently there are no tests available or known to determine the suitability, digestibility and resistance of various types of disaccharides and polysaccharides for health and disease, in adults, children or the elderly. G.M food is based on modifications of genes, characteristics and properties of different types of food related materials (example: starch). No one knows in a better and scientific form about the suitability of G.M food for consumption by people or animals including insects and other biological living creatures. This Test and Testing Machines are designed to Test the quality and resistance of Polysaccharides and Disaccharides including G.M food products, and to determine their digestibility and suitability for consumption. This will be applicable for normal healthy subjects (adults, children and elderly), subjects who suffer from various types of diseases like diabetes, gastrointestinal digestive problems, pancreatic disease etc in addition to animals. (It is an important test for Polysaccharides and Disaccharides digestibility and suitability for children and children food manufactory).

Polysaccharides Testing Machine: A apparatus, method, composition and device for testing quality and resistance of Polysaccharides based on Amylase Polysaccharides Test color changing reaction per units of time (minutes or seconds) (Iodine Test). The machine will be as an infrared electrophotometer with a sample placement chamber. The chamber is to be fitted with a removable disposable sample container. Pre-tested standard type, standard amount with standard strength sterile Testing Amylase reagent will be available for use with the test to give good results.

The machine will be calibrated with normal standard values measured and obtained from the color changing time reaction that normally occurs between Polysaccharides and iodine in the presence of amylase staining Test. The given sample to be tested will be placed in a disposable sample container and placed in the testing sample chamber. Graphic results will also be obtained and printed out from the machine. This will show the correlation between time and color changes, and results will be compared with normal standard values for the test. A software program for this test will also be available.

Polysaccharides Testing Machine (Sodium Bicarbonate Clearance Test): A apparatus, method, composition and device for testing quality and resistance of Polysaccharides based on Iodine and polysaccharides and sodium bicarbonate, color changing test reaction per units of time (minutes or seconds). The machine will be as an infrared electrophotometer with a sample placement chamber. The chamber is to be fitted with a removable disposable sample container. Pre-tested standard type, standard amount with standard strength sterile testing Iodine and Sodium bicarbonate reagents will be available for use with the test to give good results.

The machine will be calibrated with normal standard values measured and obtained from the color changing time reaction that normally occurs between Polysaccharides and iodine in the presence of Sodium bicarbonate test. The given sample of polysaccharides to be tested will be placed in a disposable sample container and placed in the testing sample chamber. The test time limited will be that of Polysaccharide Iodine stain clearance by Sodium Bicarbonate, which usually takes about one hour (accurate clearance time measurement will be provided by the machine for the standard reagents and material to be tested). Graphic results will also be obtained and printed out from the machine. This will show the correlation between time and color changes, and results will be compared with normal standard values for the test. A software program for this test will also be available.

Disaccharides Testing Machine: A apparatus, method, composition and device for testing quality and resistance of Disaccharides based on Amylase Disaccharides Test reaction per units of time (minutes or seconds). The principal and the main idea to be used in this machine will be similar to that used in the Amylase Disaccharide (sucrose) test. The machine will be similar to a glucometer but slightly bigger. It will be fitted with a sample placement chamber with a fixed sensor electrode in the base or sides, which will be connected to a disposable sample container (the idea of the disposable sample container is similar to glucometer testing strips). This will be used only once. It will be for either a continuous time reading or limited time reading. Pre-tested standard type, standard amount sterile test reagents of Amylase will be available for use with the test to give good results. Graphic results will also be obtainable and printable from the machine. This will show the correlation between time and reaction changes, and results will be compared with normal standard values for the test. A software program for this test will also be available. The idea is similar to that described in the basic (e.g., Amylase Disaccharides Test by using Sucrose, although other Disaccharides can also be used for this test).

Saliva Testing Strips: Multipurpose strip (e.g., can test for other saliva components like, pH, Specific Gravity, Proteins, etc): Saliva Testing meters with disposable strips. (This can be used for diabetes amylase treatment follow up, amylase functioning test). Measurements will be calculated from the formula: Reaction time between saliva and Sucrose per seconds/divided by the amount of sugar that the glucometer was able to read in units of time (seconds). This will range from 0.075-0.083 during the first 5 minutes and between (38.57-43.2 after 9 hours), for the normal subjects. A proper chart for the normal values will be out soon. Saliva analyzer: This will be similar to the existing blood gas machine. It will calculate saliva contents in minerals, enzymes, specific gravity, pH, etc.

Some Examples for Starch related Health Problems: Starch and Blood Clotting (Starch can cause D.V.T, P.E, etc). Starch. when eaten, some of it will be found in blood as raw uncooked intact starch. Starch in general (for example chickpeas, beans etc) if put in water they will attract water into there granules and inflate, starch is also characterised by gelatinisation (forming gel). Starch products are rich in calcium and it was also noted to calcify (undergo bone like transformation with high calcium contents) may be due to its rich calcium contents or to attraction of calcium to be deposited on it. All this if it happens in blood, this will then become like a clot. In addition to that, attracting calcium will disturb normal blood coagulation (Calcium is one of the important factors of coagulation=factor number V) and generates clots every where in the body (D.V.T=Deep Veins Thrombosis, P.E=Pulmonary Embolism, etc). It is well known in medicine that intravenous starch fluids can cause multiple clots, which can create a condition similar to that of disseminated intravascular coagulation (D.I.C). Starch deposits in tissues were also demonstrated after intravenous administration of starch infusions (Intravenous fluids) therapy.

Starch and Formation of Atheroma. Raw starch can be deposited in blood (blood walls, circulating blood), and tissues. Starch deposit on blood walls attracts calcium, fibers, lipids and other materials such as nicotine in the blood including blood cells to be deposited on it and forms what so called atheroma.

Starch and Dental Plaque. Starch also deposit on teeth and obviously attracts calcium from teeth and nicotine in addition to other materials including bacteria to give a nicotine stained dental plaque which often get cleaned by using sodium bicarbonate.

New Invented Treatment Materials and Methods for Treatment of Diabetes Mellitus and other Diseases. Saliva Therapy. The inventor discovered new Treatment for patients with salivary glands diseases. This treatment was never used before. At the moment Urine therapy is a well-known therapeutic product used for the treatment of various diseases such as high blood pressure, etc. Other body fluids and blood products for example plasma serum, albumin, etc; are also used as treatment for variety of diseases. Similarly, Digestive bacteria (What so called friendly intestinal bacteria) is also used as a drink. The inventor developed a new therapeutic pharmacological approach, which consists of the followings:

Human or animal saliva (to be collected in sterile containers). Human or animal saliva can be presented as follows: liquid medicinal syrup, medicinal solution, dry powder, tablets, capsules, ointments, vials or medicinal bottles with various amounts starting from 5, 10, 25, 50, 100, 150, 200, 250 up to thousands of mls). Different concentrations different strength of saliva components specifically the amylase with various Strength starting from 25 mg, 50 mg 100 mg and more up to even 500 mg, 1000 mg or 4000-5000 mg of amylase). It can be presented as fresh, or frozen, purified or unpurified.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification, but only by the claims.

### REFERENCES

Brooks, G. A. Anaerobic threshold: review of the concept and directions for future research. Med. Sci. Sports Exerc. 17: 22-31, 1985.
Chase P. B. and M. Kushmerick Effects of pH on contraction of rabbit fast and slow skeletal muscle fibers. Biophysics J. 53: 935-946, 1988.
Costill, D. L., F Verstappen, H Kuipers, E Janssen, and W. Fink Acid-base balance during repeated bouts of exercise: influence of HCO3. Int. J. Sports Med. 5:228-231, 1988.
Goerge, K. P. and D. P. M. MacLaren. The effect of induced alkalosis and acidosis on endurance running at an intensity corresponding to 4 mM blood lactate. Ergonomics 31: 1639-1645, 1988.
Horswill, C. A., D. L. Costill, W. J. Fink, M. G. Flynn, and J. P. Kirwan. Influence of sodium bicarbonate on sprint performance: relationship to dosage. Med. Sci. Sports Exerc. 20: 556-569, 1988.
Inbar, O., A. Rotstein, I. Jacobs, P. Kaiser, and R. Dlin. The effects of alkaline treatment on short-term maximal exercise. J. Sports Sci. 1: 95-104, 1983.
Johnson, W. R. and D. H. Black. Comparison of effects of certain blood alkalinizer and glucose upon competitive endurance. J. Appl. Physiol. 5: 577-578, 1953.
Katz, A., D. L. Costill, D. S. King, M. Hargreaves, and W. J. Fink Maximal exercise tolerance after induced alkalosis, Int. J. Sports Med. 5: 107-110, 1984.
Kindermann W, Keul J, Huber G. Physical exercise after induced alkalosis (bicarbonate or tris buffer). Eur. J. Appl. Physiol. 37: 197-204, 1977.
Linderman J, Kirk L, Musselman J, Dolinar B, Fahey T D. The effects of sodium bicarbonate and pyridoxine-alpha-ketoglutarate on short-term maximal exercise. J Sports Sci. 10: 243-253, 1992.
Linderman, J. K., T. D. Fahey, G. Lauten, A. S. Brooker, and D. Bird A comparison of blood gasses and acid-base measurements in arterial, arterialized-venous, and venous blood during short-term maximal exercise. Eur J Appl Physiol 61: 294-301, 1990
Linderman, J. K. and Fahey T D. Sodium bicarbonate ingestion and exercise performance: an update. Sports Med. 11: 71-77, 1991.
Linderman J. K. and K. L. Gosselink. Sodium bicarbonate ingestion and exercise performance: an update. Sports Med 1994 (in press).
Mainwood, G. W. and D. Cechetto. The effect of bicarbonate concentration on fatigue and recovery in isolated rat diaphragm muscle. Can. J. Pharm. 58: 624-632, 1980.
Mainwood, G. W. and P. Worsley-Brown. The effects of extracellular pH and buffer concentration on the efflux of lactate from frog sartorius muscle. J. Physiol. 250: 1-22, 1975.
McKenzie, D. C., K. D. Coutts, D. R. Stirling, H. H. Hoeben, and G. Kuzara. Maximal work production following two levels of induced metabolic alkalosis. J. Sports Sci. 4: 35-38, 1986.
Parry-Billings, M. and D. P. M. MacLaren. The effect of sodium bicarbonate and sodium citrate ingestion on anaerobic power during intermittent exercise. Eur. J. Appl. Physiol. 55: 524-529, 1986.
Roth D. A. and G. A. Brooks. Lactate and pyruvate transport is dominated by a pH gradient-sensitive carrier in rat skeletal muscle sarcolemmal vesicles. Arch Biochem Biophys. 279: 386-394, 1990.
Roth D. A. and G. A. Brooks. Lactate transport is mediated by a membrane-bound carrier in rat skeletal muscle sarcolemmal vesicles. Arch Biochem Biophys 279: 377-385, 1990;
Rupp, J. C., R. L. Bartels, W. Zuelzer, E. L. Fox, and R. N. Clark Effect of sodium bicarbonate ingestion on blood and muscle pH and exercise performance. Med. Sci. Sports Exerc. 15: 115, 1983.
Sutton, J. R., N. L. Jones, C. J. Toews. Effect of pH on muscle glycolysis during exercise. Clin. Sci. 61:331-338, 1981.
Webster, M. J., M. N. Webster, R. E. Crawford, and L. B. Gladden Effect of bicarbonate loading on exhaustive resistance exercise performance. Med Sci Sports Exerc. 25: 960-965, 1993.
Wijnen, S., F. Verstappen, and H. Kuipers. The influence of intravenous NaHCO3-administration on interval exercise: acid-base balance and endurance. Int. J. Sports Med. 5: 130-132 (Supplement), 1984.
Wilkes, D., N. Gledhill, and R. Smyth. Effect of induced metabolic alkalosis on 800-m racing time. Med. Sci. Sports Exerc. 15: 277-280, 1983.
Volkheimer G. [Persorption of microparticles] Pathologe 1993 September; 14(5):247-52. [Article in German]
Freedman B J. Persorption of raw starch: a cause of senile dementia? Med Hypotheses 1991 June; 35(2):85-7.
Volkheimer G. [The Herbst-Volkheimer effect]. [Article in German] Prokop O. Institut fur Gerichtliche Medizin des Bereichs Medizin (Charite) der Humboldt-Universitat zu Berlin, DDR. Kitasato. Arch Exp Med 1990 April; 63(1):1-6
Shaw G M, Quach T, Nelson V, Carmichael S L, Schaffer D M, Selvin S, Yang W. Neural tube defects associated with maternal periconceptional dietary intake of simple sugars and glycemic index. March of Dimes Birth Defects Foundation, California Birth Defects Monitoring Program, Berkeley, Calif. 94710, USA. gsh@cbdmp.org Am J Clin Nutr. 2003 November; 78(5):972-8.
Sebastian A, Frassetto L A, Sellmeyer D E, Merriam R L, Morris R C Jr Estimation of the net acid load of the diet of ancestral preagricultural Homo sapiens and their hominid ancestors. Department of Medicine and the General Clinical Research Center, University of California, San Francisco, Calif. 94143, USA. anthony_sebastian@msn.com Am J Clin Nutr 2002 December; 76(6):1308-16
Manary M J, Hotz C, Krebs N F, Gibson R S, Westcott J E, Broadhead R L, Zinc homeostasis in Malawian children consuming a high-phytate, maize-based diet. Hambidge KM Department of Pediatrics, Washington University School of Medicine, St Louis (MJM). Am J Clin Nutr 2002 June; 75(6):1057-61
Ullmann D, Connor W E, Hatcher L F, Connor S L, Flavell D P. Will a high-carbohydrate, low-fat diet lower plasma lipids and lipoproteins without producing hypertriglyceridemia?. Department of Medicine, Oregon Health Sciences University, Portland 97201-3098. Arterioscler Thromb 1991 July-August; 11(4):1059-67
Liu G C, Coulston A M, Reaven G M. Effect of high-carbohydrate-low-fat diets on plasma glucose, insulin and lipid responses in hypertriglyceridemic humans. Metabolism 1983 August; 32(8):750-3 33.
Olefsky J M, Crapo P, Reaven G M. Postprandial plasma triglyceride and cholesterol responses to a low-fat meal. Am J Clin Nutr 1976 May; 29(5):535-9.
Ginsberg H, Olefsky J M, Kimmerling G, Crapo P, Reaven G M. J Clin Induction of hypertriglyceridemia by a low-fat diet. Endocrinol Metab 1976 April; 42(4):729-35

1. A method of diagnosing an insulin resistant mammal comprising the steps of:
obtaining one or more samples for testing; and
determining the level of starch in the one or more samples, whereby the presence of starch correlates with insulin resistant.

2. The method of claim 1, wherein the step of testing further comprises contacting the one or more samples with one or more bicarbonates, one or more alkaline agents, one or more hexokinases, one or more oxidases, one or more amylases or combination thereof.

3. The method of claim 1, wherein the step of testing includes detecting one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof.

4. The method of claim 1, wherein the one or more samples are blood, sweat, tears, urine, saliva, cellular fluids, mucus tissues, skin, organs or combination thereof.

5. A method of detecting a polysaccharide-based disease comprising the steps of:
isolating a sample from a patient suspected of having the polysaccharide based disease; and
determining the relative level of polysaccharide in the sample.

6. The method of claim 5, wherein the step of determining the relative level of polysaccharide in the sample comprises the detection of one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof.

7. A method of treating an insulin resistant patient comprising the step of:
isolating a sample from a patient suspected of having the polysaccharide based disease;
determining the relative level of polysaccharide in the sample; and
administering a pharmaceutical formulation to the patient one or more hexokinases, one or more oxidases, one or more amylases or combination thereof.

8. The method of claim 7, wherein the pharmaceutical formulation comprises one or more enzymes, one or more compounds, one or more solutions, one or more vitamins, one or more minerals or combinations thereof.

9. The method of claim 7, wherein the pharmaceutical formulation comprises one or more bicarbonates, one or more alkaline agents, one or more hexokinases, one or more oxidases, one or more amylases or combination thereof.

10. The method of claim 7, wherein the pharmaceutical formulation is an intravenous preparation, a capsule, a tablet, a medicinal syrup, a liquid, a lotion, a spray, an ointment, a cream, a dissolvable tablet, a suppository, an effervescent tablet or combination thereof.

11. A diagnostic device comprising:
a sensor that measures the presence of one or more polysaccharides, wherein the sensor contacts a sample suspected of having polysaccharides present.

12. The diagnostic device of claim 11, wherein the sensor detects one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof.

13. The diagnostic device of claim 11, wherein the sample is combined with one or more bicarbonates, one or more hexokinases, one or more oxidases, one or more amylases or combination thereof.

14. A method of determining the total sample disaccharide/monosaccharide levels in a patient comprising the step of:
detecting an initial disaccharide/monosaccharide level in a sample;
converting one or more oligosaccharides in the sample into one or more monosaccharides; and
detecting a total disaccharide/monosaccharide level in a sample, whereby the difference between the initial disaccharide/monosaccharide level and the total monosaccharide level is the level of polysaccharides in the sample.

15. The method of claim 14, wherein the step of converting one or more oligosaccharides to one or more disaccharides or monosaccharides comprises adding one or more bicarbonates, one or more hexokinases, one or more oxidases, one or more amylases or combination thereof to the sample.

16. An pharmaceutical formulation in a dosage form for effectively decreasing polysaccharide levels comprising:
one or more agents capable of converting polysaccharides into disaccharides/monosaccharides.

17. The pharmaceutical formulation of claim 16, wherein the pharmaceutical formulation is an intaravenous preparation, a capsule, a tablet, a medicinal syrup, a liquid, a lotion, a spray, an ointment, a cream, a dissolvable tablet, a suppository, an effervescent tablet or combination thereof.

18. The pharmaceutical formulation of claim 16, wherein the one or more agents comprise one or more bicarbonates, one or more alkaline agents, one or more hexokinases, one or more oxidases, one or more amylases or combination thereof.

20. A method of determining a starch level of an individual comprising the steps of:
applying a reaction solution to a portion of the skin of the individual; and
determining the starch level in the skin with a sensor that measures the product of the reaction solution on the skin.

21. The diagnostic device of claim 20, wherein the sensor detects one or more visible wavelengths, one or more IR wavelengths, one or more Near IR wavelengths, one or more UV wavelengths, one or more fluorescence wavelengths, one or more chemiluminescence wavelengths, one or more radiation emissions, one or more FRET emissions, one or more ELISA emissions or combination thereof.

22. The method of claim 20, wherein the reaction solution comprises: iodine, water, an alcohol and a salt.

23. The method of claim 20, wherein the sensor is portable.

24. An enzyme in a dosage form for effectively decreasing polysaccharide levels, comprising:
a therapeutically effective amount of one or more enzymes capable of converting polysaccharides into disaccharides and/or monosaccharides; and
one or more pharmaceutical agents.

25. The enzyme in a dosage form of claim 24, wherein the one or more enzymes are blended with one or more agents.

26. The enzyme in a dosage form of claim 24, wherein the one or more enzymes are one or more hexokinases, one or more oxidases, one or more amylases or combination thereof.
